(19) United States
(12) Patent Application Publication
(10) Pub. No.: US 2017/0065534 A1
(43) Pub. Date: Mar. 9, 2017

(54) COMPOSITIONS AND METHODS FOR INTRADERMAL VACCINE DELIVERY

(71) Applicant: THE RESEARCH FOUNDATION FOR THE STATE UNIVERSITY OF NEW YORK, Albany, NY (US)

(72) Inventors: Katarzyna SAWICKA, Miller Place, NY (US); Sanford SIMON, Stony Brook, NY (US)

(73) Assignee: THE RESEARCH FOUNDATION FOR THE STATE UNIVERSITY OF NEW YORK, Albany, NY (US)

(21) Appl. No.: 15/308,207

(22) PCT Filed: May 1, 2015
(86) PCT No.: PCT/US15/28929

§ 371 Date: Nov. 1, 2016

Related U.S. Application Data
(60) Provisional application No. 61/987,527, filed on May 2, 2014.

(51) Int. Cl. A61K 9/70; A61K 39/35; A61K 47/32; A61K 39/02

(52) U.S. Cl. CPC A61K 9/703; A61K 39/099; A61K 39/35; A61K 47/32; A61K 2039/575

(57) ABSTRACT

The present invention relates, generally, to compositions comprising at least one antigen encapsulated within at least one fibrous polymer matrix and their use in intradermal vaccine delivery. It has been discovered that the intradermal delivery of antigens into the antigenpresenting cell-rich layers of the skin requires less antigen than traditional vaccine delivery methods and is still capable of eliciting an immune response comparable to that observed with traditional vaccine delivery methods.

### FIELD OF THE INVENTION

The present invention relates, generally, to compositions comprising at least one antigen encapsulated within at least one fibrous polymer matrix and their use in intradermal vaccine delivery.

### BACKGROUND

Human skin defends against foreign pathogens: for example, via the physical barrier posed by its architecture; or via resident antigen presenting cells (APCs) that participate in launching both humoral and cellular immune responses.

The body's ability to protect itself from foreign pathogens relies on its ability to recognize and react to such infectious materials once they are presented to the host immune cells, in a process known as the adaptive immune response.

Currently employed liquid formulations for immunization deliver antigens with a mixture of stabilizers, preservatives and components left behind by the manufacturing process, as well as adjuvants. There are four distinct classes of vaccine formulations, reflecting the form of antigen utilized: live attenuated (e.g., smallpox), killed/inactivated (e.g., whole cell), toxoid (e.g., toxin denatured with formaldahyde) and subunit vaccines. See, e.g. Baxter, D. (2007), Occupational Medicine (Oxford, England), 57(8), 552-556.

The antigen in a subunit vaccine is typically a protein component of a bacterium, virus or a DNA fragment that codes for the protein sequence and is transcribed and translated in the host. Subunit vaccines have been reported to induce both humoral and cellular responses to the administered antigen. The effectiveness of such vaccination largely depends on the antigen's immunogenicity, which is in turn a function of the antigen's size, molecular complexity, degree of “foreignness” and capacity to be cleaved into peptides by APCs.

The antigen-stimulated and activated APCs migrate to draining lymph nodes (DLNs), where the antigen is then presented to naïve T helper cells. Subsequently the antigen is presented to B cells resulting in systemic antibody production as well as priming of memory B cells. The initial production of antibodies begins to decline approximately three weeks post-primary exposure, but can be further enhanced via a second contact with the same antigen. Therefore, booster shots are strongly advocated and generally induce high concentrations of antigen-specific antibodies.

The integumentary system of the human body is its largest organ, and represents the first line of defense against foreign materials. Human skin is composed of three main layers: the stratum corneum (SC), epidermis and dermis. See FIG. 1. The 10-20 μm thick SC is predominantly made up of cornified keratinocytes, which release lipids into the intercellular spaces. The resulting brick-and-mortar structure serves as a competent, but breachable barrier. The 50-100 μm thick epidermis is mainly made up of keratinocytes and bone marrow-derived APCs known as Langerhans cells (LCs). See Glenn et al “Transcutaneous Immunization and Immunostimulant Strategies”, Immunology and Allergy Clinics of North America, 23, 2003, p 787-813; and Merad et al (2008) “Origin, Homeostasis and Function of Langerhans Cells and Other Langerin-Expressing Dendritic Cells”, Nature Reviews: Immunology, 8, 935-945.

The surveillance network of LCs occupies 25% of the skin's total surface. The cells continuously migrate out of the skin to the draining lymph nodes (DLN), but the rate of migration drastically increases upon exposure to activating stimuli such as antigens and adjuvants. The innermost of the three layers, the dermis, provides structural support to the skin. It is mostly composed of connective tissue, and is populated with its own resident APCs, the dendritic cells (DCs), as well as hair and sweat glands. Due to the presence of blood vessels the dermis has been considered the major target of transdermal drug delivery. Successful delivery of antigens to the epidermis as well as the dermis has been shown to generate cellular as well as humoral immune responses via mechanisms involving participation of major histocompatibility antigens on both classes of APCs found in the human skin. The skin's constant surveillance function and active participation in immunoprotection renders it an optimal destination for antigen delivery.

The 10-20 μm thick stratum corneum has been identified as a barrier of the body's integumentary system. Its composition and structure pose a physical barrier to penetration of liquids, large molecules and microbial agents. The transdermal route of drug delivery has been claimed to be limited to molecules smaller than 500 Da due, in part, to the impermeability of the stratum corneum, but, more important, to the skin's metabolism of macromolecular payloads. Inter-subject and inter-site differences in permeability have been attributed to variation of SC thickness and lipid content, as well as differences in skin type and age.

The “brick and mortar” lamellae of the SC that are actively involved in the management of water transport in skin have been said to resemble an accordion that flexes during lipid swelling and drying events. The external hydration of the skin induces swelling, causing reversible changes in the SC microstructure, consequently allowing passage of normally inadmissible molecules.

Recent studies compared the efficacy of a lowered (3 μg) dose intradermal immunization to that of a full 15 μg dose of the intramuscularly injected influenza vaccine formulation. The findings showed that a lower dose of the intradermal vaccine induces antibody titers that offer protection comparable to that achieved after a full dose of intramuscular vaccine. See Auewarakul, P et al “Antibody responses after dose-sparing intradermal influenza vaccination”, Vaccine, 25, 2007, p. 659-663; and Kenney et al. “Dose sparing with Intradermal Injection of Influenza Vaccine”, The New England Journal of Medicine, 2004. 351: 2295-2301. However, the method requires that approximately 2 mm of a needle penetrate the skin at a 15 degree angle followed by a slow release of the liquid into the superficial layers. Efficacy of the method requires employment of highly trained medical personnel.

Mkrtichyan, M., et al. (“Immunostimulant adjuvant patch enhances humoral and cellular immune response to DNA immunization”, DNA and Cell Biology, 27 (1), 2009, p.19-24) discloses another method of transdermal vaccine delivery consists of a patch, adjuvants and a disposable skin penetration device.

Matriano, J. A., et al. (“Macroflux Microporjection Array Patch Technology: A New and Efficient Approach for Intracutaneous Immunization”, Pharmaceutical Research, 19(1), 2002, p. 63-70) discloses a microneedle array capable of inducing an immune response in animals.

Wendorf, J. R. et al. (“Transdermal Deliver of Macromolecules Using Solid-State Biodegradable Microstructures”, Pharmaceutical Research, 2010, Published Online) discloses solid-state biodegradable microstructure (SSBMS) arrays that utilize a spring-loaded high velocity impactor to penetrate the stratum corneum with dissolvable microneedles that, upon dissolution, deliver incorporated macromolecules into the skin.

Sullivan, S. P., et al., (“Dissolving polymer microneedle patches for influenza vaccination”, Nature Medicine, 16, 2010, p. 915-920) discloses a microneedle array patch containing polyvinyl pyrrolidone (PVP) microneedles.

The present invention provides compositions and methods to immunize an animal via intradermal delivery of at least one antigen without mechanically disrupting the stratum corneum.

### SUMMARY

It has been discovered that the intradermal delivery of antigens into the antigen-presenting cell-rich layers of the skin requires less antigen than traditional vaccine delivery methods and is still capable of eliciting an immune response comparable to that observed with traditional vaccine delivery methods. Accordingly, the present disclosure provides compositions comprising at least one antigen encapsulated within at least one fibrous polymer matrix, that is used to deliver the at least one antigen intradermally to an animal without mechanically disrupting the stratum corneum.

In one aspect, the present disclosure provides compositions comprising a payload encapsulated/encased within at least one fibrous polymer matrix that is used to deliver the payload intradermally to an animal. In one aspect, the payload is at least one antigen. In one aspect, the payload is at least one antigen and at least one adjuvant.

In one aspect, the at least one antigen comprises at least one molecule used to generate a subunit vaccine from the pathogens is selected from the group consisting of influenza virus proteins, anthrax, Bordetella pertussis, human papilloma virus and combinations thereof. In one embodiment, the at least one antigen is pertussis toxin.

In one aspect of the present disclosure, the at least one fibrous polymer matrix is soluble. In one aspect, the at least one soluble fibrous polymer matrix is hygroscopic. In one aspect of the present disclosure, the at least one fibrous polymer matrix is a polymer matrix formed from at least one polymer selected from the group consisting of polyvinylpyrrolidone, polyvinyl alcohol (PVA), polyethylene oxide (PEO), polyacrylamide (PAM), and HEMA (2-hydroxyethyl methacrylate).

In one aspect, the at least one soluble hygroscopic fibrous polymer matrix is formed from polyvinylpyrrolidone. In one aspect, the average molecular weight of the polyvinylpyrrolidone is from about 100,000 g/mol to about 2,500,000 g/mol. In one aspect, the polyvinylpyrrolidone has an average molecular weight of about 1,300,000 g/mol.

In one aspect of the present disclosure, the payload is encapsulated/encased into at least one fibrous polymer matrix by mixing a solution of the payload with a solution of polymer and forming at least one non-woven membrane by electrospinning the mixture onto a substrate.

In one aspect, a first payload is encapsulated into a first fibrous polymer matrix by mixing a solution of the first payload with a solution of polymer and forming at least one non-woven membrane by electrospinning the mixture onto a substrate, and a second payload is encapsulated into a second polymer matrix by mixing a solution of the second payload with a solution of polymer and forming a second non-woven membrane by electrospinning the mixture onto the first fibrous polymer matrix. In one aspect, the second payload contains a different at least one antigen than the first payload.

In one aspect of the present disclosure, the payload is encapsulated into a fibrous polymer matrix by mixing a solution of the payload with a solution of the polymer. The payload solution may comprise from about 10% to about 90% of the mixture. The polymer solution may comprise from about 90% to about 10% of the mixture. In one aspect of the present disclosure, the payload solution is 20% of the mixture and the polymer solution is 80% of the mixture.

In one aspect, the concentration of the polymer solution is from about 0.05 mM to about 0.1 mM.

In one aspect, the concentration of the payload solution is from about 10 ng/ml to about 2000 ng/ml.

In one aspect of the present disclosure, the average diameter of the fibers is a uniform diameter from about 10 nm to about 500 nm. In one aspect of the present disclosure, the average diameter of the fibers is about 40 nm. In an alternate aspect, the average diameter of the fibers is about 72 nm.

In one aspect, a composition comprising payload encapsulated/encased within at least one fibrous matrix is attached to the skin of an animal. In one aspect, the at least one fibrous matrix increases the hydration of the stratum corneum. In one aspect, the increased hydration of the stratum corneum dissolves the at least one fibrous matrix, releasing the payload. In one aspect of the present disclosure, the increased hydration of the stratum corneum increases the permeability of the stratum corneum to the payload.

In certain aspects, the at least one fibrous matrix releases 100%, 90%, 80%, 70%, 60%, 50%, 40%, 30%, 20%, or 10% of the payload encapsulated/encased within the at least one fibrous matrix when it is attached to the skin of an animal. In certain aspects, the at least one fibrous matrix releases 100%, 90%, 80%, 70%, 60%, 50%, 40%, 30%, 20%, or 10% of the payload encapsulated/encased within the at least one fibrous matrix after 24 hours when it is attached to the skin of an animal.

### BRIEF DESCRIPTION OF THE DRAWINGS

The accompanying drawings, which are incorporated herein and form part of the specification, illustrate various embodiments of the present invention and, together with the description, further serve to explain the principles of the invention and to enable a person of ordinary skill in the art to make and use the invention. In the drawings, like reference numbers indicate identical or functionally similar elements. A more complete appreciation of the invention and many of the attendant advantages thereof will be readily obtained as the same becomes better understood by reference to the following detailed description when considered in connection with the accompanying drawings, wherein:

FIG. 1 shows a cross-section of human skin.

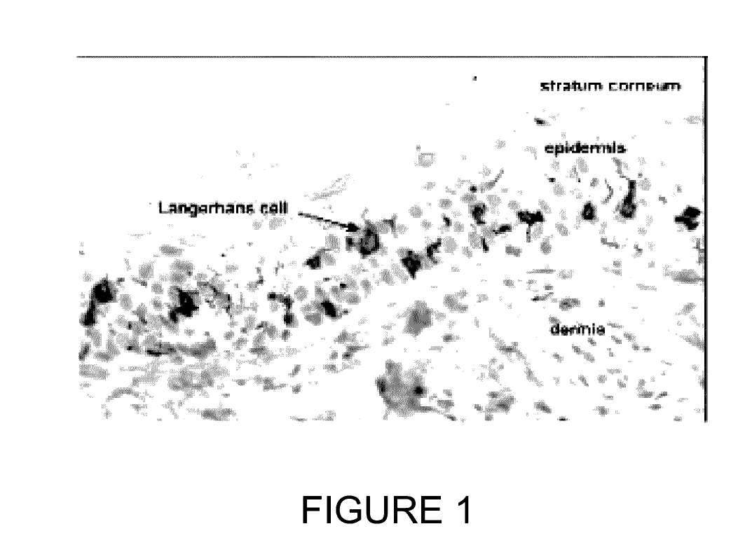

FIG. 2 shows a representative scanning electron micrograph of a fibrous polymer matrix formed via one embodiment of the methods of the present disclosure, using a mixture comprising 20% protein and 80% 0.075 mM PVP.

FIG. 3 shows scanning electron micrograph images of fibrous polymer matrices formed via one embodiment of the methods of the present disclosure, using a mixture comprising 20% protein and 80% 0.075 mM PVP, with no pause (top image); a 30 second pause (middle image); and a 60 second pause (lower image) every minute during the formation of the fibrous polymer matrices.

FIG. 4 shows representative electron micrograph images of fibrous polymer matrices formed via one embodiment of the methods of the present disclosure, using a mixture comprising 20% protein and 80% 0.05 mM PVP (left column), 20% protein and 80% 0.075 mM PVP (middle column), and 20% protein and 80% 0.10 mM PVP (right column), with no pause (top row); a 30 second pause (second row); a 45 second pause (third row) and a 60 second pause (lower row) every minute during the formation of the fibrous polymer matrices.

FIG. 5 shows an array of silicon wafers arranged on the target plate of an apparatus used to form the fibrous polymer matrix via one embodiment of the methods of the present disclosure.

FIG. 6 shows one embodiment of the indradermal delivery patch of the present disclosure.

FIG. 7 shows a standard curve used to quantify the amount of antibodies produced in an animal that had been immunized according to the methods of the present disclosure.

FIG. 8 shows the amount of antibodies to pertussis toxin produced in animals that had been immunized according to the methods of the present disclosure, using one embodiment of the intradermal delivery patch of the present disclosure (“Patch”) or control (“IM”) at the times indicated. The times at which the antigen was administered to the animals are shown.

FIG. 9 shows the skin of an animal following removal of one embodiment of the intradermal delivery patch of the present disclosure.

FIG. 10 shows a schematic of the PCF Millicell apparatus utilized for the basal media Immuno Pure Horseradish Peroxidase (HRP) release experiment outlined in Example 2.

FIG. 11 shows release of payload profiles observed for the HRP/PVP matrices prepared according to the methods disclosed in Example 2, with a 0.05 mM PVP; the error bars represent the standard deviation between the three matrices dissolved for each mixture.

FIG. 12 shows release of payload profiles observed for the HRP/PVP matrices prepared according to the methods disclosed in Example 2, with a 0.075 mM PVP; the error bars represent the standard deviation between the three matrices dissolved for each mixture.

FIG. 13 shows release of payload profiles observed for the HRP/PVP matrices prepared according to the methods disclosed in Example 2, with a 0.10 mM PVP; the error bars represent the standard deviation between the three matrices dissolved for each mixture.

FIG. 14 shows the comparison of HRP deposition on the electrospinning collector determined from the bulk release study for nine combinations of enzyme to polymer outlined in Example 2; the error bars represent the standard error of the mean obtained for three different matrices dissolved for each mixture.

FIG. 15 shows the comparison of HRP release from electrospun matrices in the PCF Millicell apparatus described in Example 2 for three concentrations of polymer vehicle; the error bars represent the standard deviation between the three wafers dissolved for each mixture.

FIG. 16 shows the comparison of HRP release from electrospun matrices in the PCF Millicell apparatus described in Example 2 for three concentrations of polymer vehicle; the error bars represent the standard deviation between the three wafers dissolved for each mixture.

FIG. 17 shows the comparison of HRP release from electrospun matrices in the PCF Millicell apparatus described in Example 2 for three concentrations of polymer vehicle; the error bars represent the standard deviation between the three wafers dissolved for each mixture.

FIG. 18 shows the average HRP delivery profile for the various platforms tested on the MatTek EFT-300 tissue engineered human skin constructs, according to the methods disclosed in Example 3. The error bars represent the standard error of the mean between the samples within each group for a specific time point (n=6).

FIG. 19 shows the average percentage of HRP delivered by the various platforms tested on the MatTek EFT-300 tissue engineered human skin constructs, according to the methods disclosed in Example 3. The error bars represent the standard error of the mean between the samples within each group for a specific time point (n=6).

FIG. 20 shows the alamar Blue cell viability assay absorption at 590 nm observed after the 24-hr delivery study for two tissue samples from each test group, according to the methods disclosed in Example 3. The error bars represent the standard of deviation between the two tissue samples tested for each group (n=2).

FIG. 21 shows a comparison of HRP concentrations obtained from the electrospun HRP/PVP Matrix wafers in tissue experiment versus the control dissolution study. The error bars represent the standard error of the mean between the samples within each group for a specific time point (n=4; samples land 2 of homogenized tissue were compromised with the alamar blue assay). See Example 3.

FIG. 22 shows the average MTS viability assay absorbance reading after the 24-hour PT delivery study disclosed in Example 3 (n=4 for UTC and PTSolution; n=6 PT/PVP Matrix).

FIG. 23 is a graphical representation of absorbance over time.

FIG. 24 is a graphical representation of tissue viability.

FIG. 25 is a graphical representation of absorbance by wavelength.

FIG. 26 is a Scanning Electron microscope (SEM) image of a nonwoven nanofibrous matrix.

FIG. 27 are inverted microscope images of biopsies.

### DETAILED DESCRIPTION

For clarity of disclosure, and not by way of limitation, the detailed description is divided into the following subsections that describe or illustrate certain features, embodiments or applications of the present disclosure.

In the present disclosure the term “animal” is used to refer to a wide range of animals, including, without limitation, mammals, reptiles, aquatic animals, humans, canines, horses, felines, and livestock. Fabrication of the Intradermal Delivery Patch of the Present Disclosure

In one embodiment, the present disclosure provides an intradermal delivery patch comprising a payload encapsulated/encased by at least one fibrous polymer matrix. In one embodiment, the at least one fibrous polymer matrix is hygroscopic. In one embodiment, the at least one fibrous polymer matrix is coated with a hygroscopic agent.

In one embodiment, the present disclosure provides compositions comprising a payload encapsulated/encased within at least one fibrous polymer matrix that is used to deliver the payload intradermally to an animal. In one embodiment, the payload is at least one antigen. In one embodiment, the payload is at least one antigen and at least one adjuvant. “Antigen” as used herein, refers to a substance which provokes an adaptive immune response.

In one embodiment, the payload is encapsulated into at least one fibrous polymer matrix by forming a mixture comprising a solution of the payload and the polymer solution, forming the mixture into fibers and forming the fibers into at least one non-woven membrane on a substrate.

In one embodiment, a first payload is encapsulated/encased into a first fibrous polymer matrix by mixing a solution of the first payload with a solution of polymer and forming at least one non-woven membrane on a substrate, and a second payload is encapsulated into a second polymer matrix by mixing a solution of the second payload with a solution of polymer and forming a second non-woven membrane on the first fibrous polymer matrix. In one embodiment, the second payload is different than the first payload.

The mixture of the payload and the polymer may be formed into fibers and the fibers formed into a non-woven membrane by any method suitable of forming fibers with diameters in the nanometer range, whilst preserving the biological function of the payload that is to be encapsulated. Suitable methods include, for example, wet spinning, dry spinning, melt spinning, gel spinning, and the like. Ideally, method used to form fibers forms a membrane comprising non-woven fibers with diameters in the nanometer range, such that the membrane has a large surface area to volume ratio and a small pore size.

In one embodiment, the mixture of the payload and the polymer is formed into fibers, and the fibers formed into a nonwoven membrane using electrostatic spinning Electrostatic spinning utilizes an electric field to overcome the surface tension of a droplet formed at the tip of a needle. The charged jet is accelerated toward a grounded or oppositely charged collecting plate. Once the jet reaches the collecting plate, it deposits as a non-woven matrix of polymeric fibers with diameters ranging from a few nanometers to micron scale, depending on the characteristics of the mixture and process parameters, such as, for example, the distance travelled by the jet.

Ideally, the flight time of the jet should allow for complete solvent evaporation, thereby preventing formation of a “beads-on-a-string” morphology of the nanofibrous matrix deposited on the target. The presence of beading is presumed to decrease the surface area reactivity of the matrix. Ideally, the viscosity of the mixture of the payload and the polymer should be such that the mixture forms fibers of uniform thickness and encapsulates an amount of the payload sufficient to evoke the desired response in an animal. One of ordinary skill in the art can readily adjust the electrostatic spinning parameters to optimize formation of the fibers.

An example of an electrostatic spinning method suitable for use in the present disclosure is disclosed in U.S. Pat. No. 1,975,504.

In one embodiment, the payload is encapsulated into a fibrous polymer matrix by a method comprising the steps of:
a) mixing a solution of the payload with a solution of polymer,
b) ejecting the mixture of the payload and from a needle into an electric field, thereby forming fibers, and
c) forming a non-woven membrane by collecting the ejected mixture on a substrate.

In one embodiment, a second non-woven membrane comprising a payload encapsulated/encased within a fibrous polymer matrix is formed on a first non-woven membrane by repeating steps a) through c). The payload and/or the polymer may be the same as the first non-woven membrane.

The rate of ejection of the mixture may be readily selected by one of ordinary skill in the art. In one embodiment, the mixture is ejected at a rate of 10 μl/min.

In one embodiment, the ejection of the mixture is paused and the electric field is turned off for a period of time during the formation of the non-woven membrane. In one embodiment, the ejection of the mixture is paused and the electric field is turned off for a period of time when a given volume has been ejected. The given volume may be every 100, or 90, or 80, or 70, or 60, or 50, or 40, or 30, or 20, or 10 μl of mixture, or any particular volume between 1 and 100 μl. The period of time may be 60 sec, or 50 sec, or 40 sec, or 30 sec, or 20 sec, or 10 sec, or any particular period of time between 1 and 60 sec.

In one embodiment, the ejection is stopped and electric field is turned off for 60 seconds every 10 minutes.

In one embodiment, the average diameter of the fibers is a uniform diameter from about 10 nm to about 500 nm. In one embodiment, the average diameter of the fibers is about 40 nm. In an alternate embodiment, the average diameter of the fibers is about 72 nm.

In one embodiment, the polymer is selected from the group consisting of polyvinylpyrrolidone, polyvinyl alcohol (PVA), polyethylene oxide (PEO), polyacrylamide (PAM), and HEMA (2-hydroxyethyl methacrylate). In one embodiment, the polymer is polyvinylpyrrolidone.

In one embodiment, the average molecular weight of the polyvinylpyrrolidone is from about 100,000 g/mol to about 2,500,000 g/mol. In one embodiment, the polyvinylpyrrolidone has an average molecular weight of about 1,300,000 g/mol.

In embodiment, the payload is encapsulated into a fibrous polymer matrix by mixing a solution of the payload with a solution of the polymer. The payload solution may comprise from about 10% to about 90% of the mixture. The polymer solution may comprise from about 90% to about 10% of the mixture. In one embodiment, the payload solution is 20% of the mixture and the polymer solution is 80% of the mixture. In an alternate embodiment, the payload solution is 25% of the mixture and the polymer solution is 75% of the mixture. In an alternate embodiment, the payload solution is 30% of the mixture and the polymer solution is 70% of the mixture.

In one embodiment, the concentration of the polymer solution is from about 0.05 mM to about 0.1 mM. The polymer may be dissolved in any solution suitable for forming fibers according to the methods of the present disclosure. In one embodiment, the polymer is dissolved in ethanol.

In one embodiment, the concentration of the payload solution is from about 10 ng/ml to about 2000 ng/ml. The payload may be dissolved in any solution suitable for forming fibers according to the methods of the present disclosure. In one embodiment, the payload is dissolved in PBS.

In one embodiment, the payload is pertussis toxin, and the polymer is polyvinylpyrrolidone. In one embodiment, the pertussis toxin us used at a concentration of 62.5 ng/ml. In one embodiment, the polyvinylpyrrolidone has an average molecular weight of 1,300,000 g/mol, and is used at a concentration of 0.1 mM. In one embodiment, the pertussis toxin is mixed with the polymer at a ratio of 30% pertussis toxin and 70% polymer. In one embodiment, the final concentration of pertussis toxin is about 18.75 ng/ml in the mixture.

In one embodiment, the payload is pertussis toxin, and the polymer is polyvinylpyrrolidone. In one embodiment, the pertussis toxin us used at a concentration of 143 ng/ml. In one embodiment, the polyvinylpyrrolidone has an average molecular weight of 1,300,000 g/mol, and is used at a concentration of 0.075 mM. In one embodiment, the pertussis toxin is mixed with the polymer at a ratio of 35% pertussis toxin and 65% polymer. In one embodiment, the final concentration of pertussis toxin is about 50 ng/ml in the mixture.

In one embodiment, the payload encapsulated/encased within the fibrous matrix retains its functionality for prolonged periods, up to about 32 weeks in storage under a variety of conditions.

In one embodiment, the at least one antigen encapsulated within the fibrous matrix retains its antigenicity for prolonged periods, up to about 32 weeks or longer in storage under a variety of conditions.

Assays to determine functionality or antigenicity of the payload are readily selected by one of ordinary skill in the art. Examples include the assays disclosed in Hewlett, E. L., et al. (1983) “Induction of a novel morphological response in Chinese hamster ovary cells by pertussis toxin.” Infection and immunity, 40(3), 1198-203. Another example is the assays disclosed in Cinatl, J., et al. (2007). “The threat of avian influenza A (H5N1). Part IV: development of vaccines”. Med. Microbiol. Immunol., 196, 213-225.

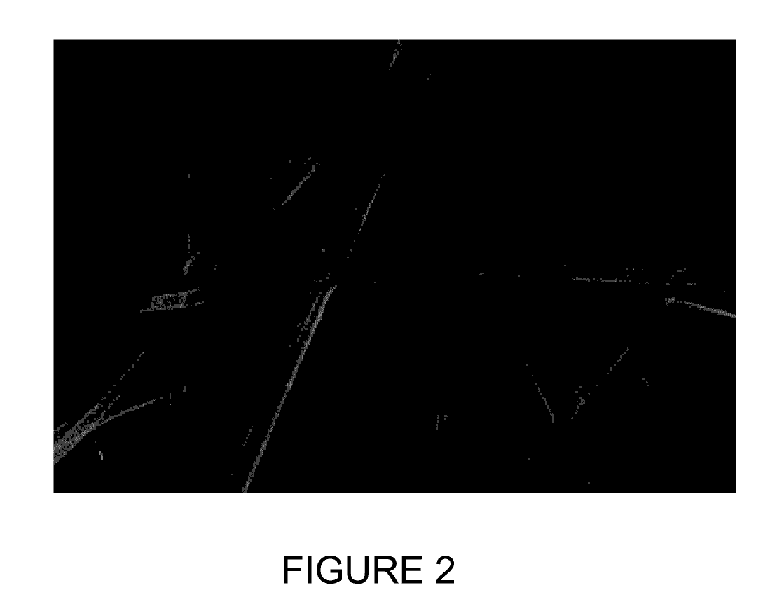

Examples of non-woven membranes formed by the methods of the present disclosure are shown in FIGS. 2-4.

In one embodiment, the payload is encapsulated into a fibrous polymer matrix by a method comprising the steps of:
a) mixing a solution of the payload with a solution of polymer,
b) ejecting 300 μl of the mixture of the payload and polymer at a flow rate of 10 μl/min from a needle into an electric field of 1.5 kV/cm, with a 10 cm distance between the needle and a target, thereby forming fibers, and
c) forming a non-woven membrane by collecting the ejected mixture on the target, wherein the target consists of an array of 4×4 mm silicon wafers.

In one embodiment, the payload is encapsulated into a fibrous polymer matrix by a method comprising the steps of:
a) mixing a solution of the payload with a solution of polymer,
b) ejecting 600 μl of the mixture of the payload and polymer at a flow rate of 10 μl/min from a needle into an electric field of 1.7 kV/cm, with a 10 cm distance between the needle and a target, thereby forming fibers, and
c) forming a non-woven membrane by collecting the ejected mixture on the target, wherein the target consists of an array of 4×4mm silicon wafers.

In one embodiment, pertussis toxin is encapsulated into a fibrous polymer matrix by a method comprising the steps of:
a) mixing a solution of pertussis toxin with a solution of polyvinylpyrrolidone,
b) ejecting 320 μl of the mixture of pertussis and polymer at a flow rate of 10 μl/min from a needle into an electric field of 1.6 kV/cm, with a 10 cm distance between the needle and a target, thereby forming fibers, and
c) forming a non-woven membrane by collecting the ejected mixture on the target, wherein the target consists of an array of 4×4 mm silicon wafers.

### Payloads Suitable for Use in the Present Disclosure

In one embodiment, the payload is at the payload is at least one antigen. In one embodiment, the payload is at least one antigen and at least one adjuvant.

In one embodiment, the at least one antigen comprises at least one molecule used to generate a subunit vaccine from the pathogens is selected from the group consisting of influenza virus proteins, anthrax, Bordetella pertussis, human papilloma virus and combinations thereof In one embodiment, the at least one antigen is pertussis toxin. In other embodiments, the at least one antigen is selected from the group consisting of antigens to the following diseases: tuberculosis, hepatitis B, polio, diphtheria, tetanus, pertussis, haemophilus influenza type b, Streptococcus pneumoniae, rotavirus, measles, human papillomavirus (HPV), Japanese encephalitis, yellow fever, tick-borne encephalitis, typhoid, cholera, meningococcus, hepatitis A, rabies, mumps, influenza and varicella.

Subunit and conjugate vaccines contain a part of a specific pathogen. Such part may comprise a protein from a specific pathogen. The protein may be isolated from the specific pathogen. Alternatively, the protein may be recombinant. The choice of protein is readily chosen by one of ordinary skill in the art.

In an alternate embodiment, the compositions of the present disclosure may be used to create vaccines for at least one protein selected from the group consisting of pertussis toxin, heamagglutinin and protective antigen.

The payload may vaccinate an animal for one, or alternatively, more than one pathogen.

### Intradermal Delivery of the Payload

In one embodiment, the intradermal delivery patch of the present disclosure increases the permeability of the stratum corneum to the payload. In one embodiment, increasing the permeability of the stratum corneum to payload enables the payload to enter the animal. The amount of the payload that enters the body is influenced by a variety of factors, such as, for example, skin type, skin thickness, temperature, hydration status, sweat gland function, the concentration of payload, and the like. Pre-treating the skin where the intradermal delivery patch of the present disclosure is to be applied may also influence the amount of the payload that enters the body. Pre-treatments can comprise cleansing, dermabrasion and the like.

In one embodiment, the intradermal delivery patch of the present disclosure increases the permeability of the stratum corneum to the at least one antigen. In one embodiment, increasing the permeability of the stratum corneum to the at least one antigen enables the at least one antigen to be taken up by antigen-presenting cells and elicit an immune response. The amount of the at least one antigen that is taken up by the antigen-presenting cells is influenced by a variety of factors, such as, for example, skin type, skin thickness, temperature, hydration status, sweat gland function, the concentration of the at least one antigen, and the like. Pre-treating the skin where the intradermal delivery patch of the present disclosure is to be applied may also influence the amount of the at least one antigen that is taken up by the antigen-presenting cells. Pre-treatments can comprise cleansing, dermabrasion and the like.

On one embodiment, the permeability of the stratum corneum is increased by increasing the hydration of the skin that contacts the intradermal delivery patch of the present disclosure. In one embodiment, the fibrous polymer attracts water, thereby increasing the hydration of the skin that contacts the intradermal delivery patch of the present disclosure. In one embodiment, the attracted water dissolves or degrades the fibrous polymer, thereby releasing the payload.

In one embodiment, the present disclosure provides a method for immunizing an animal to at least one pathogen, comprising the steps of:
a) forming at least one intradermal delivery patch comprising at least one antigen encapsulated by at least one fibrous polymer matrix,
b) attaching the at least one patch the onto the skin of the animal,
c) allowing the at least one patch to increase the hydration of the skin, thereby increasing the permeability of the skin to the at least one antigen; and
d) allowing the at least one patch to release the at least one antigen and enter the skin of the animal, thereby immunizing the animal.

In certain embodiments, the at least one fibrous matrix releases 100%, 90%, 80%, 70%, 60%, 50%, 40%, 30%, 20%, or 10% of the at least one antigen encapsulated within the at least one fibrous matrix when it is attached to the skin of an animal. In certain embodiments, the at least one fibrous matrix releases 100%, 90%, 80%, 70%, 60%, 50%, 40%, 30%, 20%, or 10% of the at least one antigen encapsulated within the at least one fibrous matrix after 24 hours when it is attached to the skin of an animal.

In one embodiment, the intradermal delivery patch is formed on a 4×4 mm silicon wafer. In one embodiment, about 37.5 ng of the at least one antigen encapsulated within at least one non-woven membrane formed on a 4×4 mm silicon wafers. In an alternate embodiment about 80 ng of the at least one antigen encapsulated within at least one non-woven membrane formed on a 4×4 mm silicon wafers.

At least one intradermal delivery patch may be applied to the skin of an animal. The at least one intradermal delivery patch may be covered by an occlusive dressing whilst applied to the skin of the animal. In one embodiment, the at least one intradermal delivery patch may applied to the skin of an animal for 24 hours.

In certain embodiments, the animal is contacted with at least one intradermal delivery patch for 24 hours, then subsequently contacted with another at least one intradermal delivery patch. The subsequent treatment may be 21 days after the first contact.

The permeability of the skin to the payload may be enhanced by maximizing the surface area of contact between the patch and skin. A lack of stable adhesion of the delivery patch to the skin can result in detachment, thus breaking the permeation process and delivery of the payload. The at least one intradermal delivery patch may be attached to the skin of the animal by any suitable method. Such methods may include, for example, via placing an adhesive bandage over the at least one intradermal delivery patch.

Each of the embodiments and examples discussed herein of the disclosed intradermal delivery patch can be effectively utilized with or without any mechanical disruption (e.g. pricking, scratching, etc.) to the skin the patch is to be placed on. Further, the disclosed patch can operate effectively without the addition of external hydration to the patch because the disclosed patch can achieve suitable permeability of the skin.

Also, each of the embodiments and examples discussed herein can be configured to deliver antigens having any molecular weight that can suitably pass through an animal's skin, such as a human's skin. Examples of the molecular weight of the antigens include about 30 kDa or greater, about 40 kDa or greater, about 50 kDa or greater, about 60 kDa or greater, about 70 kDa or greater, about 80 kDa or greater, about 90 kDa or greater, about 100 kDa or greater, about 110 kDa or greater, about 120 kDa or greater, about 130 kDa or greater, about 140 kDa or greater, about 150 kDa or greater, about 160 kDa or greater, about 170 kDa or greater, about 180 kDa or greater, about 190 kDa or greater, about 200 kDa or greater, about 220 kDa or greater, about 240 kDa or greater, about 260 kDa or greater, about 280 kDa or greater, about 300 kDa or greater, about 350 kDa or greater, about 400 kDa or greater, about 450 kDa or greater or about 500 kDa or greater.

The present invention is further illustrated, but not limited by, the following examples.

### EXAMPLES

### Example 1

### Immunization of Animals to Pertussis Toxin Using the Compositions of the Present Disclosure

Intradermal Delivery Patch Formation: The polymer carrier solution consisted of polyvinyl pyrrolidone (PVP) M.W. approximately 1,300,000 g/mol [Sigma-Aldrich, Milwaukee, Wis.] prepared as a 0.075 mM solution in absolute ethanol (EtOH). Lyophilized pertussis toxin, obtained from List Biological Labs [Campbell, Calif.], was reconstituted to a stock concentration of 143 ng/μl in sterile Hank's Buffered Saline Solution (HBSS) [Gibco, Grand Island, N.Y.]

The electrospinning solution with a final PT concentration of 50 ng/μl, was prepared as follows: 35% by volume PT (143 ng/μl)−4900; and 65% by volume PVP (0.075 mM in EtOH)−9100.

The electrospinning solution was magnetically stirred for five minutes before being placed into a glass syringe, and placed into the programmable syringe pump. The PT/PVP coating was generated by ejecting 1000 μl of the electrospinning solution at a flow rate of 20 μl/min into an electric field of 1.7 kV/cm with a 10 cm distance between the needle and the target. The process was paused for 1 minute at 10 minute intervals to enhance deposition.

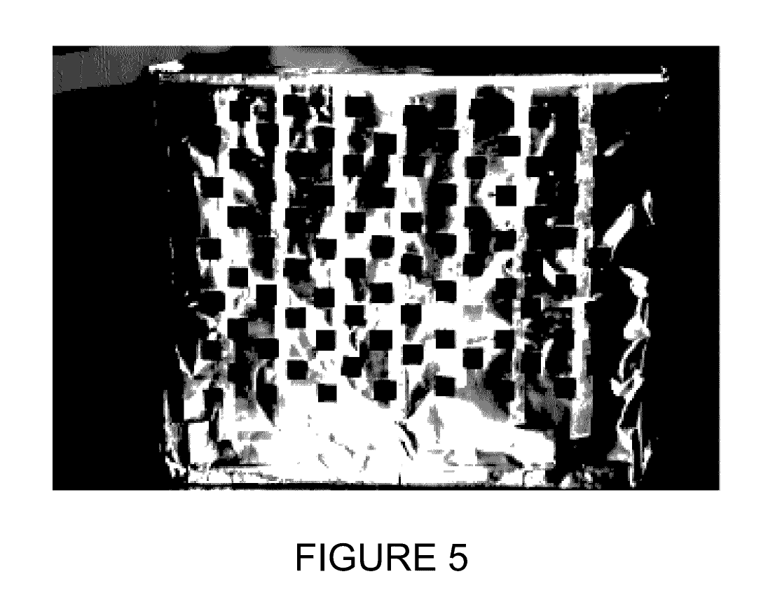

Based on a conservative estimate for deposition, approximately 80% of the electrospun solution accumulates on the aluminum foil-wrapped 8×10 cm target plate. Since each of the 4×4 mm silicon wafers [Silex Microsystems, Boston, Mass.] utilized for the patch occupies 0.2% of the target's total surface area, it can be deduced that approximately 0.2% of the total electrospun solution was be deposited on each of the eighty wafers attached to the foil. See FIG. 5. The amount of PT collected on each wafer has been correlated to its surface area. The 4×4 mm wafer occupies 0.2% of the collector's area. The parameters utilized for electrospinning resulted in an 80% deposition rate of material on the collection plate. A total of 40,000 ng of PT accumulated on the entire foil, indicating that each silicon wafer coating contained approximately 80 ng of PT.

The PT-Patch was constructed by adhering 12 PT-PVP wafers to a Tegaderm transparent film dressing [3M, Saint Paul, Minn.]. The waterproof dressing allows for water vapor and oxygen exchange while providing a barrier to bacterial and viral contamination. The wafers were attached in a staggered formation of three rows with four wafers each, to maximize adhesion between the skin and patch. The patch was then returned to its packaging and stored in an airtight plastic container with desiccant until it was attached to the animal's back, approximately one hour later.

Animal Model: The animal model chosen was the Sprague-Dawley rat. 10-week old Sprague Dawley male rats from Charles River Laboratories (Wilmington, Mass.), weighing approximately 300 grams, were randomly selected and divided into two groups of five: animals in the first group received PT via intramuscular injection while animals in the second group received PT administered via the “PT-Patch.” To aid in identification, the animals were marked at the base of their tails and housed individually throughout the course of the study. Proper handling, housing, care, and standard rodent food was given to the animals according to the guidelines posted by Stony Brook's Institutional Animal Care and Use Committee (IACUC) and Stony Brook's Division of Laboratory Resources (DLAR).

Control—Intramuscular Injection: The intramuscular injection solution was prepared via a dilution of stock PT to a final concentration of 9.6 ng/μl of PT in sterile HBSS; each dose of 100 μl, containing 960 ng of PT, was injected directly into the quadriceps muscle of the hind leg using a 25-gauge needle. Blood Collection and Immunization: The animals were sedated using isoflurane inhalation anesthesia for each blood collection and vaccine administration. All of the animals were immunized on days 0 and 21. Blood samples were obtained using the retro-orbital method prior to the initial immunization, and again on days 14, 28 and 42. All whole blood samples were collected into 1 cc blood collection vials, and centrifuged at 3000 rpm for 10 minutes. Approximately 200 μl of plasma was obtained from each sample and was divided into 20 μl aliquots and stored at −20° C. to allow for simultaneous determination of antibody titers.

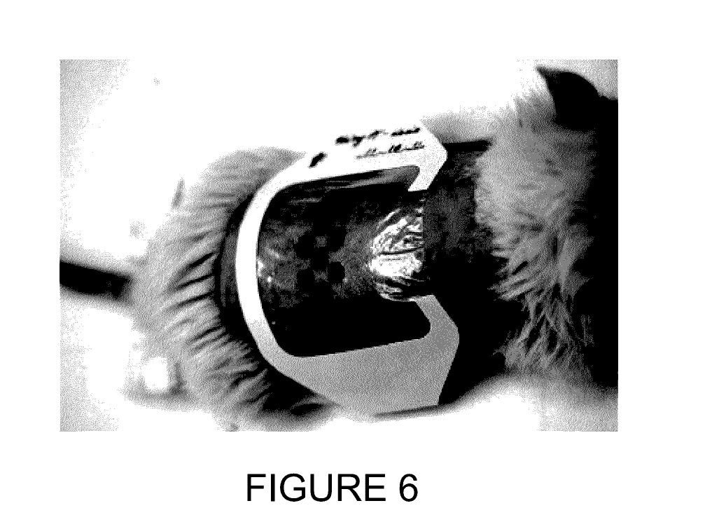

The PT-patches were applied immediately after blood collection on days 0 and 21. The dorsal surface of each animal receiving a PT-patch was shaved with an electric razor to remove bulk of the hair, and further depilated with an application of Nair hair-removing cream “for sensitive skin.” The skin was then cleaned with wet gauze to remove any excess cream. Prior to application of the PT-Patch, the skin surface was cleaned with 70% isopropyl alcohol wipes and allowed to dry. See FIG. 6. After application to the dorsal surface, the patch was bandaged over with Vetwrap to protect it from the grooming efforts of the animals. To further discourage the grooming efforts we taped over the animals' feet to prevent them from removing the patch. The patch, bandage and tape were left in place for 24 hours after application.

After removal of the patch, the site was wiped clean with sterile saline to remove any residual material. A secondary goal of this study was to evaluate any local responses of the skin at the application site upon removal of the patch and bandage. The immunization site was photographed and qualitatively examined for signs of edema and erythema at the site within fifteen minutes after patch removal, as well as 1, 3, and 7 days after each administration.

Serology: The body's ability to produce neutralizing antibodies to a delivered immunogen has been established as a highly relevant measurement of effective delivery of the antigen to the immunocompetent layers of the skin. The sera obtained at the previously mentioned time points were analyzed for a humoral response in rats by a custom designed enzyme linked immunosorbent assay (ELISA).

The ELISAs were carried out in 96-well plates [BD Biosciences, Franklin Lakes, N.J.]. The wells were coated with 2000 aliquots of either PT antigen used for immunization (150 ng/well) or a serial dilution of control Rat IgG antibody in PBS at the concentrations of 0, 0.005, 0.075, 0.150, 0.225, 0.300, 0.375, 0.450 and 0.600 ng/μl, and incubated overnight on a plate shaker at room temperature. Next day, the antigen was removed, and after three washes with PBS the plate wells were blocked with 2000 of 20% BSA-PBS for three hours on a plate shaker. Once the blocking step was completed, the plate was washed three times with 0.01% Tween-PBS and once with PBS. The next step was to allow capture of any anti-PT antibodies in the serum samples, which was achieved by a two hour incubation of 100 μl of a 1:100 serum dilution in 4% BSA-PBS added to each washed PT well. The contents of the plate were once again removed and the plate was washed three times with 0.01% Tween-PBS and once with PBS. Quantitation of the captured anti-PT antibodies was achieved by incubating each well with 100 μl of peroxidase-conjugated AffiniPure Goat Anti-Rat IgG (H+L) [Jackson ImmunoResearch Laboratories, West Grove, Pa.] at a dilution of 1:5000 in 4% BSA-PBS for one hour. The plate was once again washed and then incubated for 30 minutes with 1000 of stabilized tetramethylbenzidine (TMB) substrate [Pierce; Rockford, Ill.], followed by an addition of 100 μl of 2 M sulfuric acid to stop the reaction and convert the product to its final yellow color. Measurements of optical density were taken at a wavelength of 650 nm for the kinetic reaction of the conjugated peroxidase with TMB over a time course of 30 minutes, followed by a second read at 450 nm for the end point.

Statistical Analysis: All of the data used for statistical evaluations were checked for normality and equality of variance, using the Shapiro-Wilk and Levene's tests, followed by appropriate tests for the given experiment. All statistical tests were performed using SPSS software [IBM, Somers, N.Y.] assuming a significance level of p<0.05.

Each test group consisted of five animals. To assess the changes of IgG titer levels across time points within each group we employed the nonparametric Friedman's test. To compare two time points, the Wilcoxon Signed Rank Test was used.

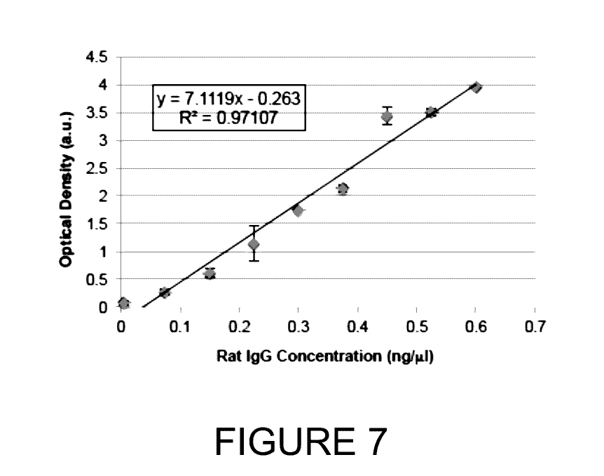

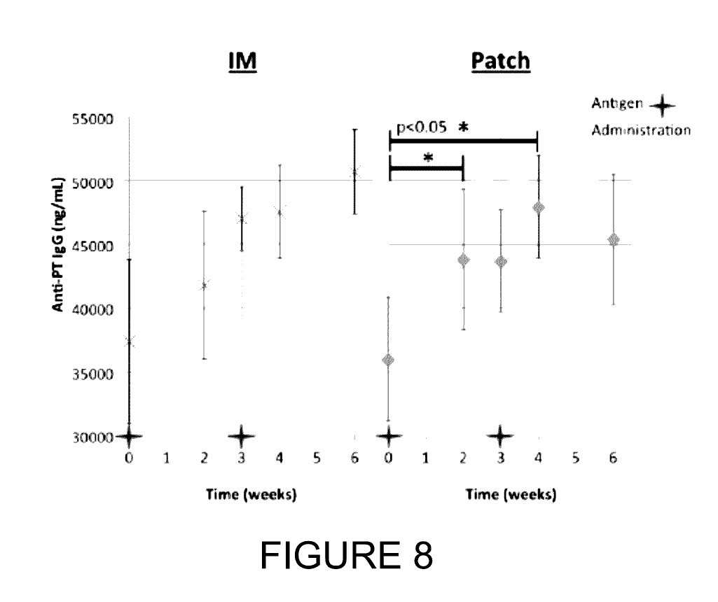

Results: The reference Rat IgG ELISA standards produced a linear regression line with the formula of y=7.119×−0.263 and a coefficient of determination of 0.97107. See FIG. 7. The obtained formula was then utilized to convert the optical density values observed for the study sera to calculate Anti-PT IgG concentrations. See FIG. 8. The data were corrected for the baseline values for any anti-PT IgG detected prior to the initial immunization. The error bars depicted show the variation between the duplicate wells of the ELISA.

An overall comparison of the time points within each group indicated that the values obtained for the PT-Patch (Friedman's test, p=0.05) but not the IM injection (p=0.28) changed significantly over time. To understand which time points differed from each other we performed the Wilcoxon Signed Rank Test. The PT-Patch values increased significantly from week 0 to week 2 and from week 0 to week 4 (p<0.05). The PT-Patch values also tended to increase from week 0 to 3, 2 to 4, and 3 to 4 (p=0.08). A trend was also observed for the intramuscular injection group; IgG levels tended to be higher after 6 weeks compared to 3 weeks (p=0.08).

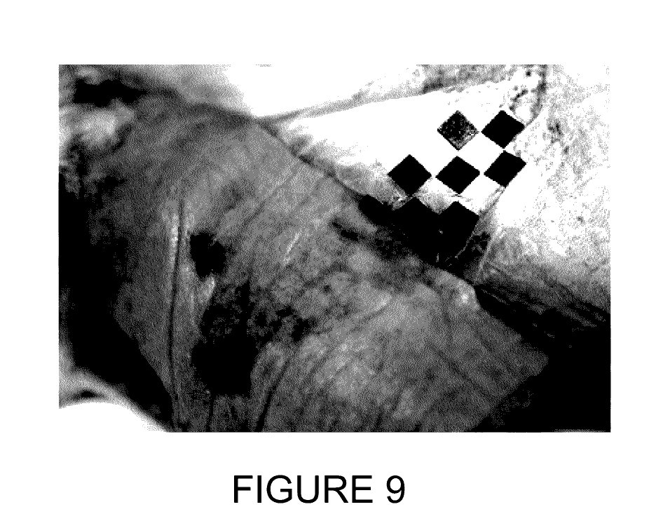

The secondary goal of this study was to evaluate the local skin tolerance to the occlusive patch. Images were taken immediately after patch removal. See FIG. 9.

The nanocomposite PT-Patch administered for 24 hours on days 0 and 21 evoked a significant humoral response in the Sprague-Dawley rat model with mean anti-PT IgG concentrations comparable to those triggered by intramuscular injection of the antigen. The change in anti-PT antibody concentrations increased significantly in response to each PT-Patch administration, and were more robust than those observed for IM animals. Clinically employed wound dressing, Tegaderm adhesive film, used as the occlusive backing on the PT-Patch, appeared to leave the animal skin with limited temporary irritation, but the silicon wafers onto which the PTPVP nanocomposite was deposited provoked no visible reaction.

Each application of the PT-Patch resulted in a robust response, faster than that triggered by the standard route of inoculation. Furthermore the increase in the antibody levels observed for the Patch group achieved statistical significance 2 weeks after the initial administration, and 1 week after the booster administration at 3 weeks. The rapid response observed in the rats receiving the PT Patch may reflect the involvement of the large numbers of APC residing in the epidermis of the animals that are preferentially mobilized by the intradermal delivery protocol to capture antigens.

### Example 2

### Formation of the Compositions of the Present Disclosure

Materials and Methods: Immuno Pure Horseradish Peroxidase (HRP) obtained as a salt-free lyophilized powder from [Pierce; Rockford, Ill.] was reconstituted in phosphate buffered saline (PBS) at a concentration of 0.598 units/pl.

The HRP standard curves were obtain through dilutions of the stock solution with PBS.

Polyvinylpyrrolidone (PVP) of M.W. 1,300,000 from Sigma-Aldrich [St. Louis, Mo.] was dissolved at 0.1 mM, 0.075 mM or 0.05 mM concentrations in absolute ethanol, allowing for a variety of viscosities to maximize the inclusion of payload within the matrix. The electrospinning solutions tested varied in percent volume of the polymer solution as presented in Table 1.

TABLE 1

| Payload Volume | | | |
|---|---|---|---|
| % | Polyvinylpyrrolidone (PVP) 1,300,000 MW | | |
| 20% | 80% | 80% | 80% |
| | 0.05 mM | 0.075 mM | 0.1 mM |
| 25% | 75% | 75% | 75% |
| | 0.05 mM | 0.075 mM | 0.1 mM |
| 30% | 70% | 70% | 70% |
| | 0.05 mM | 0.075 mM | 0.1 mM |

All of the outlined solutions were constructed with various dilutions of HRP with PBS to obtain the final HRP concentration of 0.00208 units of enzyme per microliter of the mixture.

Formation of Matrices: The electrospinning solutions outlined above were magnetically stirred for five minutes before being placed into a 2 cc glass syringe, Popper & Sons [New Hyde Park, N.Y.], and placed into the programmable syringe pump [KD Scientific; Holliston, Mass.]. The HRP/PVP coating was generated by ejecting 300 μl of the given electrospinning solution at a flow rate of 10 μl/min into an electric field of 1.5 kV/cm with a 10 cm distance between the needle and the target. The matrix was collected onto an 8×10 cm block covered with aluminum foil. Three mats were obtained for each of the nine solutions.

Bulk Solubilization and Release of Payloads: The highly hygroscopic nanocomposite matrix containing HRP as the payload was employed to assess the dissolution of the polyvinylpyrrolidone matrix and release of the incorporated macromolecular payload. The aluminum foil containing the electrospun matrix was dissolved by a triple wash with 5 ml of PBS, collecting liquid into a reagent reservoir that was placed onto a gentle rocker to allow for mixing of the solutions. The time dependency of dissolution was evaluated by collection of sequential 100 μl wash at 0.5, 1, 1.5, 2, 2.5, 3, 4, 5, 6, 8 and 10 minutes, and analyzed for the HRP concentration present.

Payload inclusion analysis: The colorimetric HRP assay was carried out in 96-well microplates, where 10 μl duplicates were mixed with 100 μl stabilized TMB (3,3′,5,5′-Tetramethylbenzidine) from Pierce [Rockford, Ill.] and recorded the kinetic reaction at 650 nm. The reaction was stopped after 30 minutes by addition of 100 μl of 2.0 M sulfuric acid and endpoint values were obtained at 450 nm. A standard curve of known HRP concentrations was generated simultaneously to allow for accurate quantification of the HRP content in unknown samples. The optical density measurements were obtained using the Versa Max Microplate Reader [Molecular Devices, Sunnyvale, Calif.].

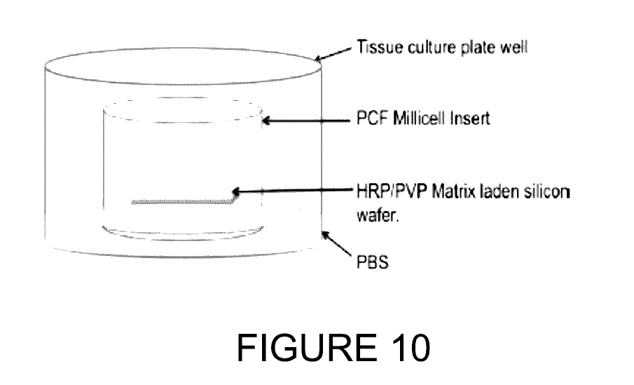

Basal Media Solubilization of HRP/PVP Matrix: The electrospun matrix for each of the nine HRP/PVP solutions outlined above was collected onto three 4×4 mm silicon wafer [Silex, Boston, Mass.]. Each wafer was faced down onto the 0.4 μm PCF Millicell® tissue sample holder [Millipore, Billerica, Mass.]. The device's polycarbonate filter bottom made basal contact with 500 μl of the subjacent aqueous medium (PBS), and allowed for slower payload release. See FIG. 10.

The tissue holder units were relocated to a fresh volume of 5000 of PBS at 5, 30, 60 and 120 minutes followed by a final wash of the wafer in the absence of the Millicell unit. The collected buffer was analyzed for functional HRP with the colorimetric assay explained above. Control wafers were dissolved in equal volumes to obtain the average deposition of HRP on the wafers.

SEM Analysis: Polyvinylpyrrolidone (PVP) (Mw˜1,300,000; Sigma Aldrich, St. Louis, Mo.) solutions were prepared in a mixture of 80% by volume ethanol and 20% of 0.1% bovine serum albumin (BSA) phosphate buffered saline (PBS). The composition of the solutions aimed to imitate the surface tension, viscosity and ionic charge of the HRP/PVP mixtures examined. The three polymer concentrations used were 0.05, 0.075, and 0.10 mM. See Table 2.

TABLE 2

| Flow Rates | Voltage | Distance | Volume | Pause Intervals |
|---|---|---|---|---|
| 5 μl/min | 15 kV | 10 cm | 250 μl | No pause |
| 10 μl/min | | | | 30 sec |
| 20 μl/min | | | | 45 sec |
| | | | | 60 sec |

The process was paused by stopping the fluid ejection and turning off the voltage supply. Each condition was run in multiples, and a representative area from each sample was examined with the scanning electron microscope (SEM).

Statistical Analysis: All of the data used for statistical evaluations were checked for normality and equality of variance, using the Shapiro-Wilk and Levene's tests, followed by appropriate tests for the given experiment. All statistical tests were performed using SPSS software [IBM, Somers, N.Y.] assuming a significance level of p<0.05.

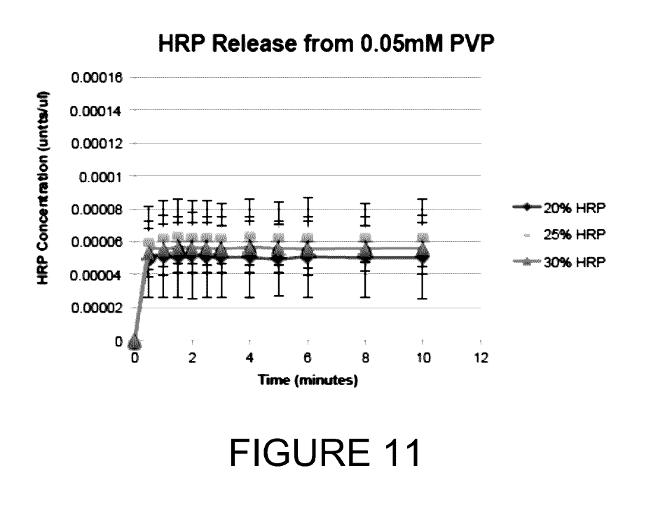

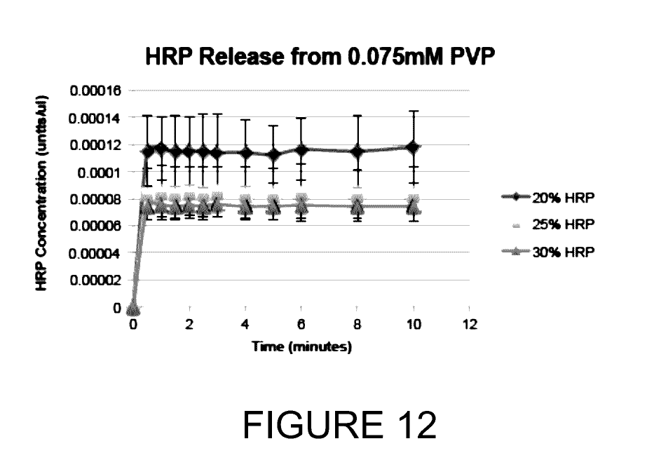

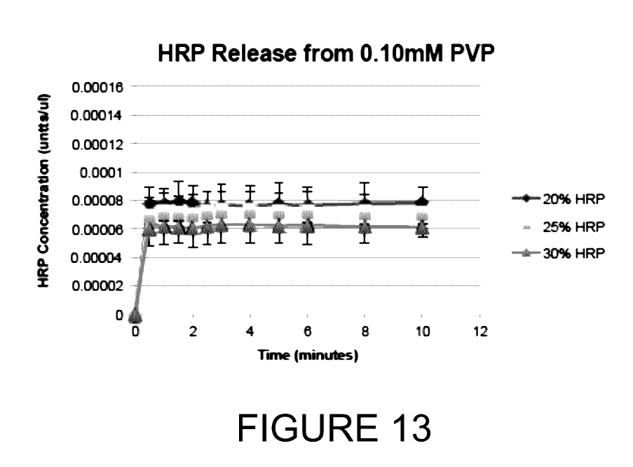

Results: Bulk Dissolution: The release profiles for the nine enzyme-polymer mixtures were represented in concentrations of HRP as well as the percentage of the expected enzyme concentration. See FIGS. 11-13. The percentage of HRP deposition was calculated by relating the observed enzyme concentrations to that expected for all of the samples, 0.000125 units/μl.

An overall comparison of the different PVP/HRP compositions showed that the deposition of HRP differed significantly between conditions (p=0.05; Kruskal-Wallis test). The deposition (%) observed for the 20% HRP 0.075mM PVP mixture was significantly higher than that of the other compositions (p=0.05; Mann-Whitney test) with the exception of the 25% HRP 0.075 mM PVP mixture (p=0.13).

The goal of this study was to optimize the electrospinning process parameters for synthesis of nanofibrous matrices of a model macromolecule, horseradish peroxidase. By performing a bulk dissolution study we were able to illustrate the preservation of enzymatic activity of the enzyme throughout the process of electrospinning. The highly hydrophilic nature of PVP aided in the instantaneous release of HRP from the electrospun biocomposite matrix regardless of its composition within the first 30 seconds. The concentration of the extracted enzyme was evaluated as a percentage of the total amount of protein electrospun. We established the most favorable mixture for HRP stability to be 20% HRP-80% 0.075 mM, with the highest percentage of functional HRP extracted from the matrix. The bolus release of HRP in the presence of a large volume, led to the development of a novel release study, that utilized a 0.4 μm PCF Millicell® tissue sample holder to create a barrier between the electrospun coating and available moisture. The developed setup resulted in a steady release of the enzyme from the electrospun wafers during a 2 hour time period. The final aim of this study was to evaluate the matrix morphology through the use of SEM. We were able to successfully encapsulate HRP within matrices of PVP that presented an average diameter of 40 nm, presenting with continuous 3D porosity and an advantageous surface area to volume ratio. Lastly we were able to validate the effect of brief pausing on the matrix morphology i.e. bead formation.

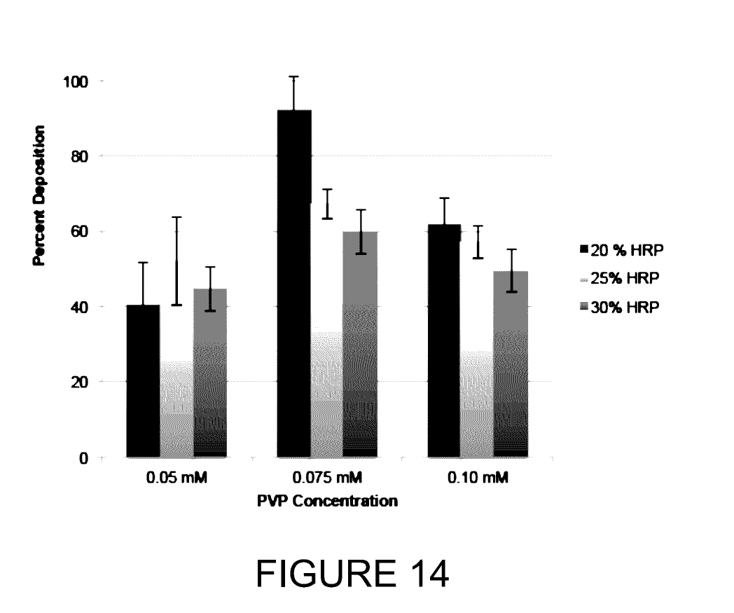

The successful incorporation of biological molecules into electrospun fibers without apparent loss of activity has been widely reported within the last decade. It was therefore hypothesized that despite the harsh process of electrospinning, HRP would maintain its catalytic function. Through the use of a colorimetric assay where HRP oxidizes a chromogenic substrate we were able to confirm preservation of as much as 92% of enzyme activity with the most advantageous combination of polymer to enzyme 20% HRP-80% 0.075 mM (see FIG. 14). It appeared that the lowest polymer concentration, and therefore the least viscous of the mixtures resulted in the smallest percentage of deposition or function retention. The two terms are used interchangeably throughout the study, as it is not clear if the loss of enzyme is due to its denaturation or limited deposition on the electrospinning collector. The sizeable error bars depicted represent the variation between three mats of the same composition. The main source of deviation came from the third, consecutively electrospun mat for each of the nine conditions (data not presented). Analysis of enzymatic activity of the solution after the completion of three mats showed a dramatic drop in HRP activity. The loss of enzymatic activity is said to be a result of the prolonged, approximately two hour, exposure to the polymer solvent, absolute ethanol. The other possibility for this drop may be due to the exposure to room temperature for the duration of the experiment.

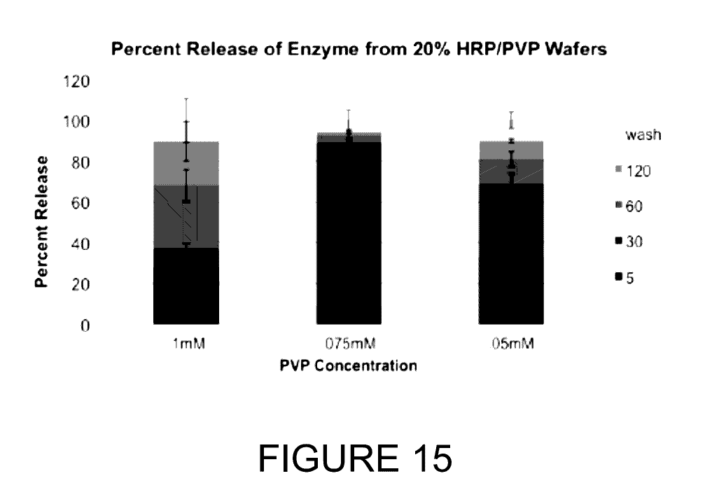

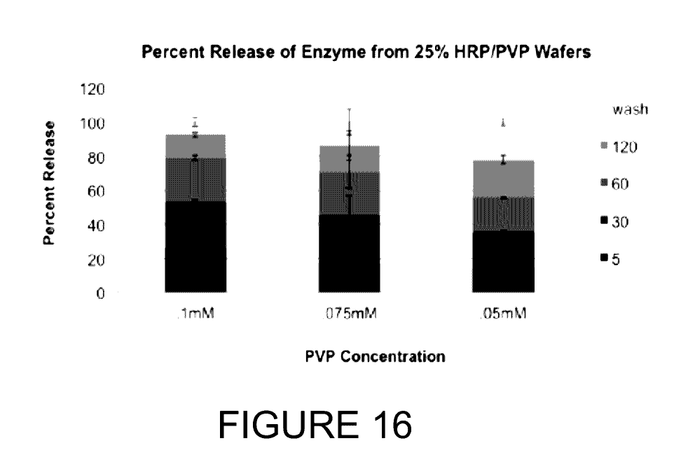

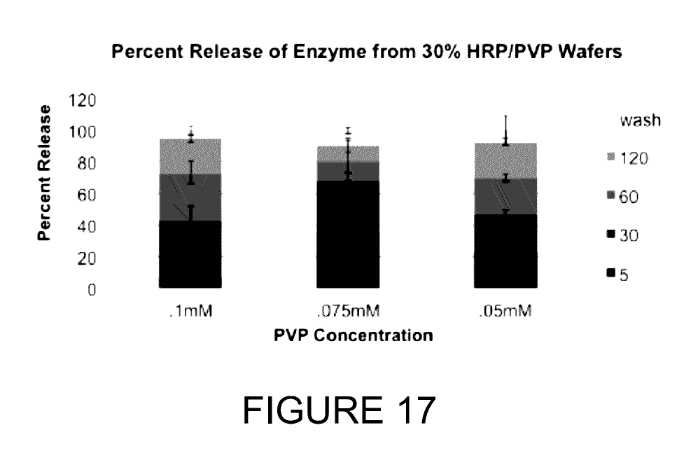

The bolus release of enzyme from the electrospun mats in the presence of 5 ml of PBS may not adequately showcase the surface solubility of the electrospun nanofibrous matrices when applied directly to the skin, as is expected to occur upon close contact with the SC. Therefore a novel approach to matrix dissolution, utilizing the 0.4 μm PCF Millicell® tissue sample holders, presented an improved model for electrospun matrix dissolution. The polycarbonate filter of the unit posed a permeable barrier between the buffer and the nanofibrous matrix, which allowed for a controlled release of the enzyme, as depicted in FIGS. 15-17. The optimal combination of HRP/PVP determined above to be 20% HRP-80% 0.075 mM PVP, exhibited the most robust excretion of the enzyme in the first 30 minutes, where the other compositions required double that time to achieve a similar amount of resolubilized enzyme. The control wafers dissolved in buffer determined that on average the electrospun matrix deposited approximately 0.18% of the total deposition on the 4×4 mm wafers, which occupy 0.2% of its total surface area.

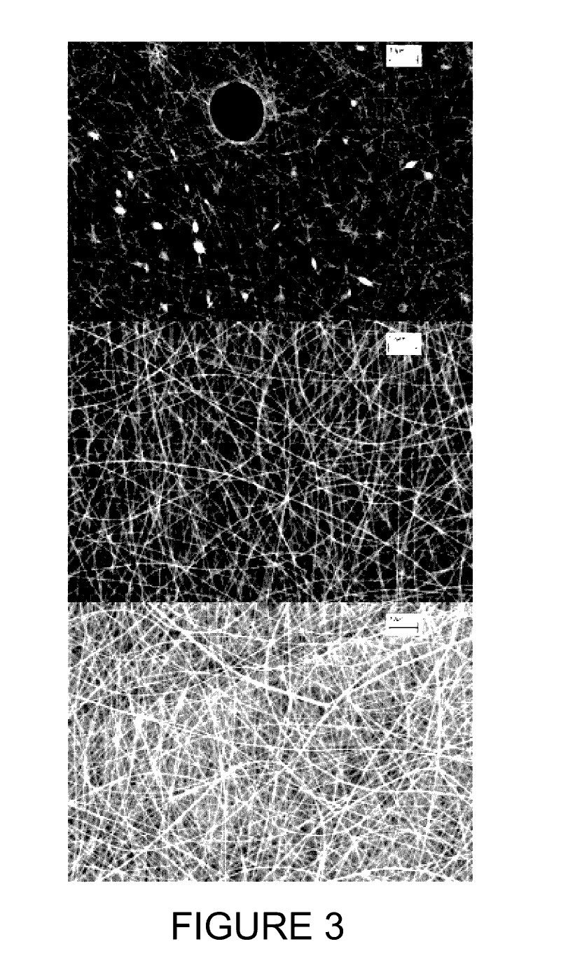

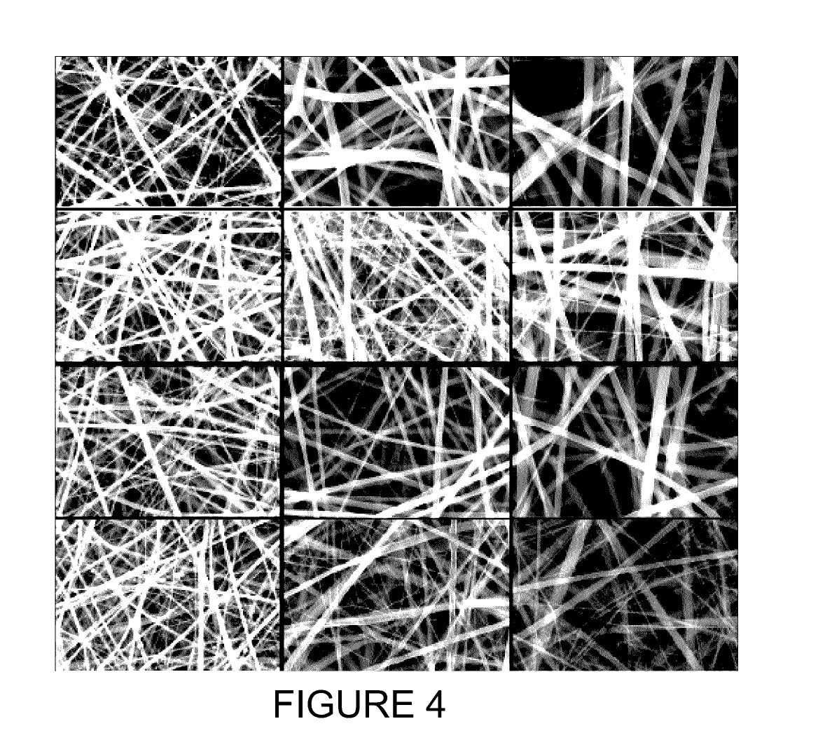

One of the chief aims of this study was optimize the electrospinning technique to obtain maximized payload inclusion into random, nonwoven nanofibrous mats of the highly biocompatible polymer, polyvinylpyrrolidone (PVP). We found that the hygroscopic polymer's capacity to readily absorb moisture is further enhanced by the high surface area to volume and mass ratios observed for electrospun mats. Although the obtained biocomposite matrices confirmed the nanofibrous morphology, the large proportion of aqueous solvent for enzyme stability resulted in a large density of beading, thus limiting the surface area available for a given electrospun coating. To address the morphological drawback, we proposed to introduce an interval pause to the process. The effect of process stoppage was examined through the employment of the optimal proportionality of the enzyme to polymer, where HRP was substituted for BSA. The introduction of the process pause was shown to result in a decrease of the average fiber diameter for a given polymer concentration, (see FIG. 3). Another significant consequence of introducing a pause was the elimination of the beads-on-the-string morphology, (see FIG. 4). At this point it is not fully understood why a brief break in the matrix production has an effect on the final morphology. It was hypothesized that the solvent vaporization during the jet flight from the tip of the needle towards the collector may influence the local humidity that limits further liquid evaporation. By introducing the pause, we allowed the present humidity to fully evaporate or condense and deposit at the bottom of apparatus, away from the jet path. This simple alteration of the procedure was employed in all further studies.

### Example 3

### Intradermal Delivery of Payload

Materials and Methods—HRP Delivery—Solutions: Polyvinylpyrrolidone (PVP) of M.W. 1,300,000 from Sigma-Aldrich was dissolved at 0.075 mM concentration in absolute ethanol. Immuno Pure Horseradish Peroxidase (HRP) obtained as a salt-free lyophilized powder from Pierce [Rockford, Ill.] was reconstituted in phosphate buffered saline (PBS) at a concentration of 0.598 units/μl.

The electrospinning solution consisted of 80% of polymer solution by volume and 20% of the enzyme solution, to obtain the final concentration of 0.1196 units/μl. The HRP solution used as a positive control in the delivery study was obtained by further diluting the stock with PBS to a final concentration of 0.10 units/μl.

HRP/PVP Matrix: The electrospinning solution consisting of 20% HRP by volume was magnetically stirred for five minutes before being placed into a glass syringe, and placed into the programmable syringe pump. The HRP/PVP coating was generated by ejecting 600 μl of the electrospinning solution at a flow rate of 10 μl/min into an electric field of 1.7 kV/cm with a 10 cm distance between the needle and the target.

The matrix was collected onto an 8×10 cm block covered with aluminum foil to which 24 4×4 mm silicon wafers [Silex Microsystems, Boston, Mass.] were attached. The wafers were positioned as 12 pairs of closely located wafers to allow for an estimation of HRP deposition for each wafer used in the tissue study. A “control” experiment was carried out, where one of the paired wafers was dissolved in 500 μl of tissue culture media in the absence of a skin model. The control wafer media was harvested and analyzed for HRP content at two time points of 4 and 24 hours.

To aid with efficient loading of HRP/PVP matrix wafers onto the tissue models, we employed ultra-clear microcentrifuge tubes with a diameter of 8 mm that were cut to a height of approximately 1 cm. The tubes were sterilized overnight with a 70% ethanol soak and subsequently dried in the sterile environment of the tissue culture hood for approximately a half hour, by being placed onto stainless steel screws.

Full Thickness Himan Skin Constructs: To study the HRP delivery profiles we employed the EpiDerm Full Thickness (EFT-300) tissue models from MatTek Corporation. The tissue units were handled under sterile conditions and according to the supplier's directions. The adjustments of the maintenance media volume from 1 mL to 0.5 mL and switching from a 6-well to a 12-well plate for the duration of the delivery experiment were the only alterations from the supplied protocol. The 24 tissue samples were divided into four groups of n=6. The test groups consisted of Untreated Controls (UTC), HRP Solution, HRP/PVP Solution and HRP/PVP Matrix. To eliminate variability between study groups, all of the liquid antigen formulations were constrained to a volume of 10 μl. to limit any hydrostatic contribution to payload entry. The HRP/PVP Matrix samples were attached to plastic vials with crazy glue, and placed on top of the tissue holders, thus beginning the 24 hour study.

The tissue experiment had three time points of 1 hour, 4 hours, and 24 hours, where the tissue insert was moved to a fresh 0.5 mL of maintenance media, while the previous time point medium was collected and analyzed using the colorimetric HRP assay.

To compare the integrity of the tissue samples between the test groups and the untreated control group at the conclusion of the 24 hours delivery study, we employed the alamar blue assay. Since the viability assay's interaction interfered with the colorimetric HRP assay, we used only the first two tissue samples from each group. The alamarBlue reagent [LifeTechnologies Corp, Grand Island, N.Y.] was diluted down to 10% concentration with the maintenance media utilized in the delivery study, and incubated the tissues in the reagent/media mix at 37° C. for four hours. At the conclusion of the four hour incubation, the liquid was harvested and analyzed in duplicates. The results were measured using the CYTOFLUOR 2300 [Millipore, Billerica, Mass.] fluorescence reader with an excitation wavelength of 530/25 nm and an emission filter of 590/35 nm The obtained values were corrected for blank alamarBlue/media mix background signal.

To extract the HRP delivered into the tissue models we homogenized the tissue after the 24 hours media harvest. Samples 3-6 for each test group were cut out of the holders using scalpels and placed into 1.5 mL centrifuge tubes together with 500 μl of fresh tissue culture media. With the use of a tooth polisher and a pellet pestle [Sigma-Aldrich, Milwaukee, Wis.] the tissue was pulverized. The mixture was centrifuged until all of the debris settled and the liquid portion was collected and analyzed using the previously described HRP assay.

PT Delivery—Solutions: 50 μg of lyophilized, salt-free, 117 kDa Pertussis Toxin (PT) [List Biological Labs, Campbell, Calif.] was reconstituted in 0.80 ml of sterile Hank's buffered saline (HBSS) resulting in a concentration of 62.5 ng/μl PT in HBSS.

The PT standards employed as positive controls for the cell culture assay were made with HBSS dilutions of stock PT to obtain a range of concentrations from 5 to SOng/ml PT in HBSS.

Polyvinylpyrrolidone (PVP) of M.W. 1,300,000 from Sigma-Aldrich was dissolved at 0.10 mM concentration in absolute ethanol.

The electrospinning solution consisted of 70% of the polymer solution by volume and 30% of the PT stock solution, to result in a final concentration of 18.75 ng/μl.

The PT Liquid formulation was diluted with HBSS to a final concentration of 1.875 ng/μl, delivering 20 μl per tissue.

PT/PVP Matrix: The electrospinning solution consisting of 30 % PT by volume was magnetically stirred for five minutes before being placed into a glass syringe, and placed into the programmable syringe pump. The PT/PVP coating was generated by ejecting 1250 μl of the electrospinning solution at a flow rate of 10 μl/min into an electric field of 1.5 kV/cm with a 10 cm distance between the needle and the target.

The matrix was collected onto a target covered with aluminum foil that 52 4×4 mm silicon wafers [Silex Microsystems, Boston, Mass.] were adhered to. From previous studies, it has been established that approximately 80% or 1000 μl of the ejected electrospinning solution would deposit onto the 8×10 cm collector, and therefore each of the wafers would collect 37.5 ng PT as each occupies 0.2% of the total collector surface area. To confirm the presence of biologically functional PT within the PT/PVP matrix a “control” study was conducted. 1, 2, 4 and 6 wafers were dissolved in 1.5 ml of EPI-100 media. The wafers employed in the tissue study were loaded onto sterilized microcentrifuge tubes as described above.

Human EpiDerm Constructs: To study the novel PT/PVP matrix's ability to deliver the toxin into and through the outer most layers of human skin we employed the EpiDerm™ (EPI-200) tissue model obtained from MatTek Corporation. As previously noted the EPI-200 model consists of multilayered/highly differentiated normal, human-derived epidermal keratinocytes as well as a stratified SC. The tissue samples were handled under sterile conditions and according to the supplier's directions. The two changes to the supplied protocol were, adjustment of the maintenance media volume from 1 ml to 0.75 ml and switching from a 6-well to a 12-well plate for the duration of the delivery experiment. The 14 tissue samples were divided into three groups. The test groups consisted of Untreated Controls (UTC) n=4, PT Solution n=4, and PT/PVP Matrix n=6. The PT/PVP Matrix wafers were attached to plastic vials with crazy glue, and placed on top of the tissue holders, beginning the 24-hour study. The basal maintenance media was collected 24 hours after the delivery sample application.

We employed the MTS cell viability assay to compare the integrity of the tissue units between the PT loaded groups to that of the untreated control group. The MTS reagent CellTiter 96® AQueous One Solution [Promega, Madison, Wis.] was diluted down to 15% concentration with HBSS. 300 μl aliquots of the diluted solution were placed in each well of a 24-well plate. After triple rinses with PBS and blot drying on a paper towel the tissue units were placed into the MTS, and incubated at 37° C. for three hours. At the conclusion of the incubation all the tissue units were collected for the homogenization process, described below. The optical density of the resultant MTS reagent was measured at a wavelength of 490 nm using the Versa Max Microplate Reader [Molecular Devices, Sunnyvale, Calif.]. The samples were analyzed in duplicates and the obtained values were corrected for blank MTS/HBSS mix background signal.

To extract the PT delivered into the tissue models, we pulverized the tissue after the 24 hours media harvest. All the samples were cut out of the holders using scalpels and placed into 1.5 mL glass vials together with 750 μl of fresh EPI-100 media. The tissue was homogenized with the use of a tooth polisher and a pellet pestle [Sigma-Aldrich, Milwaukee, Wis.]. The ground tissue was allowed to settle, the liquid portion was collected and analyzed using the CHO cell assay.

CHO Cells: Confluent CHO-K1 ATCC CCL-61 cells were trypsinized and plated into four sterile 96-well flat bottomed cell culture plates at a concentration of 2.7×10⁴ cells/cm², equivalent to 0.20 ml of 3.78×10⁴ cells/ml, except for the peripheral rows and columns with HBSS to avoid the edge effect previously observed for CHO cells cultured in 96-well plates. The cells were allowed to adhere over a 24-hour incubation period, at the end of which the original medium was replaced with duplicate 200 μl aliquots of the samples listed below:
PT Standards: 0.0, 6.25, 12.5, 25.0, 37.5, 50.0ng/ml in EPI-100.
Dissolved control wafers, where triplicates of 1, 2, 3, or 4 wafers were dissolved in 1.5ml of EPI-100.
Untreated control medium 1 through 4, at time points 24, and homogenized.
PT liquid formulation tissue sample medium 1 through 4, at time points 24, and homogenized.
PT/PVP Matrix tissue sample medium 1 through 4, at time points 24, and homogenized.

The plates were examined for the characteristic CHO cell clumping in the presence of functional PT at 12-hour intervals for a 48-hour time period. The microscopic observations were recorded at 48 hours post dosing, using a qualitative grading range from 0 to 2, where a grade of zero indicated no observable clumping.

Statistical Analysis: All of the data used for statistical evaluations were checked for normality and equality of variance, using the Shapiro-Wilk and Levene's tests, followed by appropriate tests for the given experiment. All statistical tests were performed using SPSS software [IBM, Somers, N.Y.] assuming a significance level of p<0.05.

Results: The HRP assay was employed to obtain an HRP standard curve, which would allow the conversion of sample absorbance into HRP units in the given sample. With the correlation coefficient of R2=0.97802, the standard curve trend line equation was utilized to establish the HRP concentration of the experimental samples.

The delivery profiles were represented in both concentrations of HRP as well as percentage of the expected enzyme concentration. The variation between the concentrations of HRP delivered was caused by the varying amount of enzyme delivered through the different preparations tested, as depicted in Table 3.

TABLE 3

| Sample | HRP Solution | HRP/PVP Solution | HRP/PVP Matrix |
|---|---|---|---|
| Description | HRP in PBS | 20% HRP-80% | 20% HRP-80% |
| | | PVP solution | PVP electrospun |
| | | | mat |
| Concentration | 0.10 units/ml | 0.1196 units/ml | N/A |
| HRP Units | 1.0 unit | 1.196 units (10 μl) | 4 × 4 mm wafer |
| Predicted | (10 μl) | | 0.1148 units |

An overall comparison of the delivered HRP concentrations (%) revealed a statistically significant difference between the groups for the following time points: 1 hour, 4 hours, and 24 hours using the nonparametric Kruskal-Wallis test (p=0.02, p=0.001, p=0.006respectively). The pulverized tissue samples were not different from each other (p=0.37). To understand which of the groups differed from each other at a given time point we performed the Mann-Whitney U test. The results revealed that the HRP Solution group had higher scores than the HRP/PVP Solution group at 1, 4, and 24 hours after delivery (all p-values=0.004). When we compared the HRP Solution group to the HRP/PVP Matrix we observed higher scores for the solution for the first two time points (p=0.055 and p=0.004, respectively). At the 24-hour time point, the percent delivery of enzyme solution to that of the polymer matrix showed no significant difference. Finally, the Mann-Whitney U test comparing the HRP/PVP Matrix to the HRP/PVP Solution yielded higher scores for the Matrix at 4 (p=0.004) and 24-hours (p=0.01) after delivery.

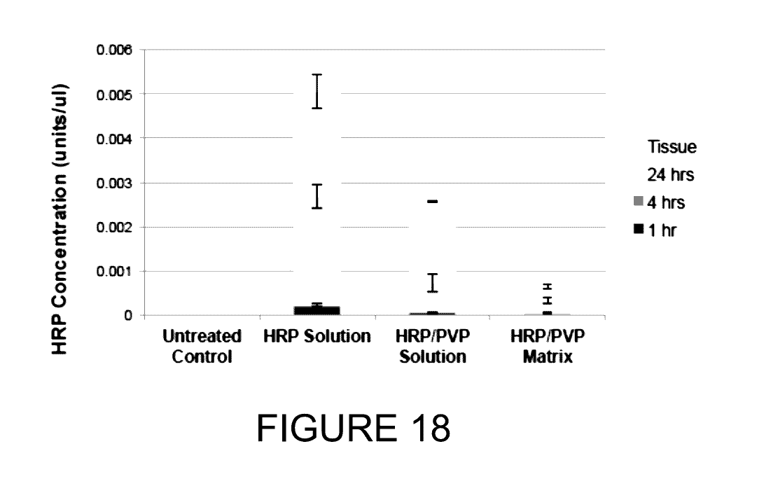

To confirm that the tissue viability was not compromised during the delivery we compared the absorbance values across the groups using the nonparametric Kruskal-Wallis test. The values were not significantly different from each other (p=0.14), suggesting that the tissue was not compromised during the delivery. See FIGS. 18-21.

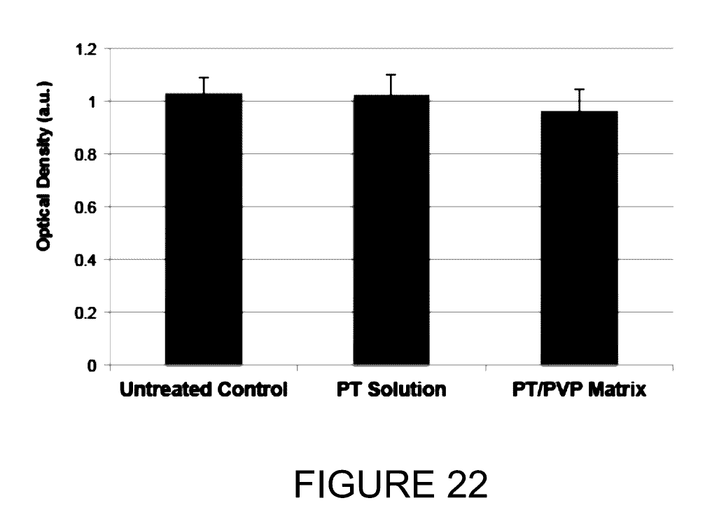

PT Results: The results are shown in FIG. 22, and Tables 4 and 5. To confirm that the results obtained for the PT/PVP Matrix were not due to chance we employed the nonparametric binomial test. Results showed that the score of 1 observed for the PT/PVP Matrix rejects the null hypothesis that the scores of 0 or 1 were equally likely to happen (p=0.03). Although the PT Liquid Formulation data are in the same direction, the n=4 wasn't enough to reach significance using the binomial test.

TABLE 4
The level of CHO cell clumping observed 48 hours after dosing
with duplicates of the control solutions of PT standard as well
as the electrospinning solution (n = 2).

| PT | PT Standard | | PT/PVP | |
|---|---|---|---|---|
| Concentration | Solution | | Standard Solution | |
| (ng/ml) | Average | SD | Average | SD |
| 0 | 0 | 0 | 0 | 0 |
| 6.25 | 1 | 0 | 1 | 0 |
| 12.5 | 1 | 0 | 1 | 0 |
| 25 | 2 | 0 | 2 | 0 |
| 37.5 | 2 | 0 | 1.5 | 0.7 |
| 50 | 2 | 0 | 2 | 0 |

TABLE 5
The level of CHO cell clumping observed 48 hours after dosing with the
media collected from the PT delivery EPI-200 tissue experiment.

| Tissue | 24 Hours | | Homogenized | | # Tissue |
|---|---|---|---|---|---|
| Sample | Average | SD | Average | SD | Units |
| Untreated | 0 | 0 | 0 | 0 | 4 |
| Control | | | | | |
| PT Liquid | 1 | 0 | 1 | 0 | 4 |
| Formulations | | | | | |
| PT/PVP | 1 | 0 | 1 | 0 | 6 |
| Matrix | | | | | |

To confirm that the tissue viability was not compromised during the delivery we compared the absorbance values across the groups using the nonparametric Kruskal-Wallis Test. The values were not significantly different from each other (p=0.28), suggesting that the tissue was not compromised during the delivery.

To further enhance the qualitative assay, we adapted an interval scale of grading, from 0 to 2. The lowest score meant no visible cell clusters, the score of 1 was given wells that presented some clusters, and finally the score of 2 was given to wells, where clusters were the predominant cell morphology. To ensure the integrity of the CHO cell assay, each plate contained duplicate stock PT dilutions ranging from 6.25-50.0 ng/ml, where the concentrations of 6.25 and 12.5 ng/ml resulted in a score of 1, while the higher PT concentrations were assigned the score of 2. The grading scale also served as a rough correlate for the amount of PT extracted from the subjacent media as well as the homogenized tissue units.

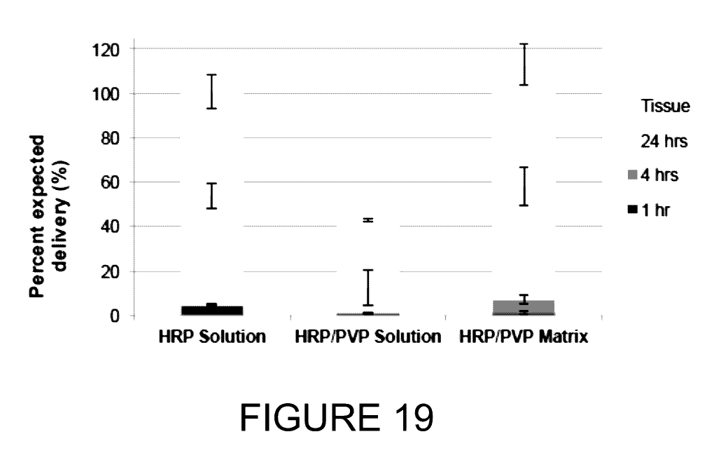

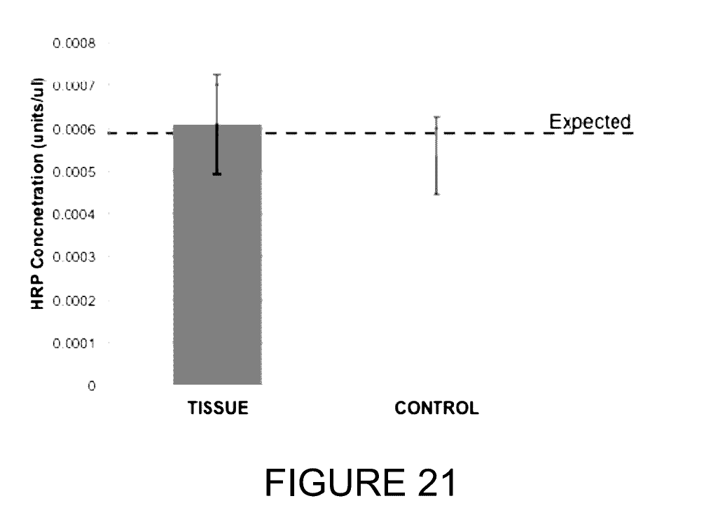

The sensitive and cost effective quantitative assay of HRP functionality was the chief proponent for its utility as a primary model for the in vitro study of intracutaneous delivery of macromolecules to human skin. Delivery of the enzyme was analyzed in terms of the amount delivered as well as percentage of the expected deposition. The stacked graphs of FIGS. 18 and 19 depict group averages for each time point. Although both liquid formulations were expected to deliver equal amounts of the HRP units recovered from the tissue media as well as the pulverized tissue for the electrospinning solution group was half of the pure enzyme formulation. One of the potential explanations of this finding is the viscous nature of the polymer solution, which may have resulted in a repeated pipetting error for the small volume of 10 μl . Another possibility is that the organic solvent employed in the polymer formulation may have hindered the functionality of the active enzyme present in solution over the 24 hour incubation period. Many of the commercially available skin patches are composed of dried polymer-based gels, but as seen in this study as a viscous solution the gelous load was not successful at delivering a large percentage of the load. Due to the size limitation posed by the tissue chambers the HRP/PVP Matrix samples were constrained to applying only one wafer per tissue, thus constituting only 10% of the enzyme load found in either liquid formulation. However the analysis in terms of percent delivery for every time point provided a more insightful picture of the patch's efficacy in delivering viable enzyme into and through the full thickness human skin model. Both liquid formulations as well as the solid-state patch showed very limited (<6%) delivery of the enzyme within the first four hours of the study. Thus implying the tissue model's ability to pose a substantial permeation barrier such as expected of health human skin. The combined percentages of enzyme delivered at the conclusion of the 24-hour incubation were 54%, 12% and 58% for the HRP Solution, HRP/PVP Solution and HPR/PVP Matrix respectively. With the bulk of the remaining enzyme recovered from the pulverized tissue, except for that of the HRP/PVP Solution units. The 24-hour incubation of the pure HRP solution showed minimal drop in its enzymatic activity/concentration. The secondary goal of this experiment was to evaluate the validity of pairing the matrix wafers by position on the collector. The pairing system proved a reliable indicator for the amount of enzyme collected on each wafer, it further confirmed the estimated 0.2% of total deposition collecting on each wafer, as seen in FIG. 21. The control HRP/PVP Matrix study confirmed that the dissociation of the nanocomposite material and release of the enzyme in the absence of tissue occurs almost completely within the first four hours upon contact with the media.

To confirm the presence of viable PT released from the PT/PVP Matrix we dissolved a number of control wafers and dosed the liquid onto the CHO cells along side the other test samples. Based on the 0.2% per wafer deposition model, the expected amount of PT collection was calculated to be 37.5 ng per wafer resulting in a PT concentration of 25 ng/ml in EPI-100 media, CHO clustering of 2. The score of 1, observed for the PT Solution and PT/PVP Matrix formulations used in the tissue delivery study suggests levels of recovered PT lower than the expected 37.5 nanograms. The similar levels of clustering observed for both formulations which were prepared to present equal amounts of PT to the tissue samples, suggests that the reduction in the amount of active PT was an experimental error. One of the potential sources of error may have been the pulverization step. Due to PT's affinity to plastic surfaces, we employed glass vials, which did not allow for centrifugation and instead required slow sedimentation of the homogenized tissue. The subjective nature of the CHO cell assay assessment presents a potential for misgraded samples due to human error.

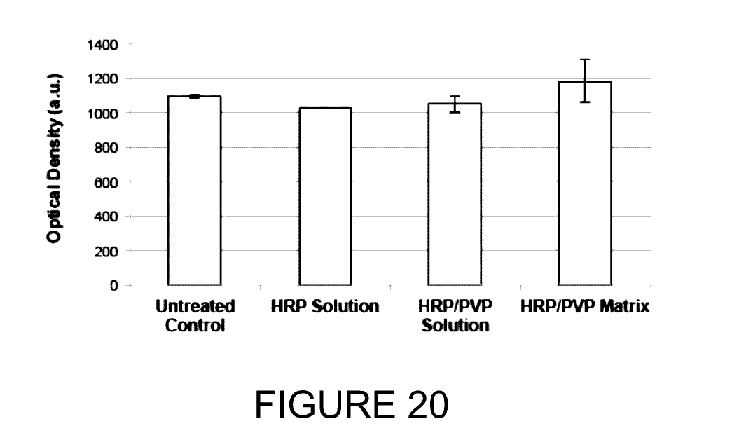

To evaluate the cytotoxic effects the novel immunization patch had on human skin constructs we employed two different cell viability assays. The integrity of the EpiDermFT™ models was assessed at the conclusion of the 24-hour HRP delivery study using the alamar blue assay. The assay measured the presence of fluorescent resorufin product that results from the reduction of resazurin, the active ingredient of the alamar blue assay, by the healthy cells. The comparison of fluorescence levels detected for the untreated control units to that of enzyme laden tissue samples, both liquid formulations as well as the novel HRP/PVP matrix was presented in FIG. 20, showed no significant decline in the absorbance across the test groups. Viability of EpiDerm™ units employed in the PT delivery study was assessed using the non-lytic MTS assay. Metabolic activity of the cells was measured through the reduction of tetrazolium salt to the colorimetric product, formazan. As demonstrated in FIG. 22, the absorption levels across all of the test groups were unvarying, between the untreated units to those of laden tissue samples, both in the liquid formulation as well as the novel PT/PVP matrix. The results of the non-lytic viability assays employed in both of the delivery studies suggest that the surface contact with the novel immunization patch presents no cytotoxic effect on the human skin cells present in the MatTek tissue samples.

The novel nanofibrous matrix developed as an innovative intradermal immunization patch was shown to successfully transport two biologically functional macromolecules into and across the commercially available tissue engineered human skin models. 24-hour administration of the HRP containing patch resulted in over half of the enzyme load penetrating the full thickness skin model, without a detectable drop in tissue viability as measured by the alamar blue assay. 24-hour administration of the PT laden patch resulted in detectable amounts of the antigen present in the media subjacent to the epidermis human skin model, as well as in the homogenized tissue. Both in vitro delivery studies render the patch an attractive painless method of macromolecule delivery that circumvents the need for physical disruption of the skin's barrier. Utilization of engineered human skin equivalents offered a high throughput in vitro permeability comparison without the complications arising from natural variability of cadaveric human skin samples reported in experiments using Franz cells as experimental assay tools. The quality control of the skin models and limited variability between tissue units offers a more practical strategy for patch optimization than would be possible with individual Boyden chamber-like units utilized with cadaveric skin. The in vitro approach also reduces reliance on animal testing and furthermore lowers the costs of acquiring and maintaining the test subjects.

### Example 4

### Transdermal Delivery of Payload

Transdermal drug delivery of HRP was compared between a dried HRP/PVP solution and an electrospun matrix. In this comparison equally sized pieces of aluminum foil were coated with either an electrospun matrix (n=6) or a dried HRP/PVP solution (n=6) and placed on tissue engineered full thickness human skin constructs. The electrospun matrix was a fibrous polymer matrix of PVP encapsulating the HRP.

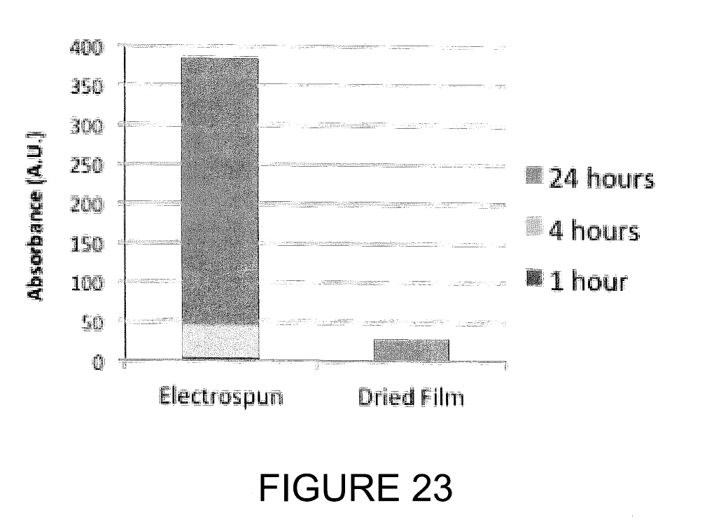

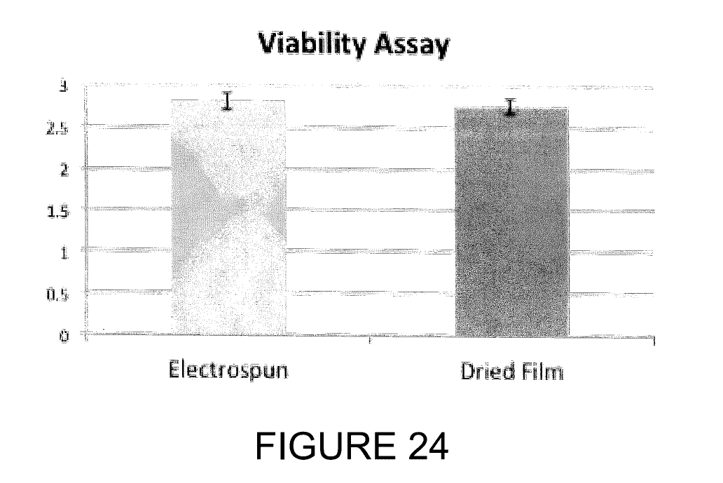

As can be seen from FIG. 23, the absorbance of the HRP from the electrospun matrix is significantly higher at one hour, four hours and at twenty four hours as compared to the dried HRP/PVP solution. Further, as shown in FIG. 24, tissue viability did not appear to be compromise by either treatment, as confirmed with the MTT assay.

### Example 5

### Delivery of Payload for Treatment or Suppression of Allergic Responses

Repeated exposure to allergens within dermal layers has been shown to lead to production of allergen—specific IgG/IgG₄ antibodies. Thus, diverting the immune response from the large and rapid IgE to a slow and weak IgG mediated response. Typically allergen mixtures are administered via subcutaneous injections. Approximately 200 administrations are given through a time period of four years. The doses are designed to increase 40—fold throughout the treatment schedule, and call for close monitoring of patient at the initial stages of dose increases.

The disclosed intradermal delivery patch can be configured to combine the efficacy achieved by allergen injections with the convenience of an efficient method for delivery.

To entrap allergens into a fibrous polymer matrix, a relatively viscous polymer solution of a hygroscopic polymer or polymer mix can be prepared. For example, the polymer solution can be prepared as a solution of 1,300,000 g/mol PVP dissolved in an organic solvent. Optionally, the polymer solution can include an aqueous co-solvent at concentrations of about 0.01 mM to about 10 mM.

The treatment allergen mixtures are produced by Greer Immunology in aqueous solvent and can be adapted into patch formation using the same six dilution gradients (shown in the table below), with each one resulting in six consecutive concentrations, such as provided by the manufacturer.

TABLE 6

| Dose # | Dose Volume (ml) | Concentration |
|---|---|---|
| 1 | 0.05 | 1 BAU/ml |
| 2 | 0.10 | Through serial ten fold |
| 3 | 0.20 | Dilutions of stock |
| 4 | 0.30 | 10,000 BAU/ml |
| 5 | 0.40 | |
| 6 | 0.50 | |

BAU—bioequivalent allergy unit.

The patch for allergies to cats can be formulated in six consecutive tenfold dilutions of the stock allergen solution obtained at 10,000 BAU/mL. In order to accommodate the increasing volume of each dilution, the patch size can be adapted to represent each dose outlined in Table 6.

At the lowest dilution of 1 BAU/mL, doses of 0.05, 0.1, 0.2, 0.3, 0.4, and 0.5 mL correspond to 0.05, 0.1, 0.2, 0.3, 0.4 and 0.5 BAU of the allergen. This system was designed so that each 4×4 mm single crystal silicon square could equal the lowest dose amount required for administration, which allows for an increased number of squares on a patch to achieve a specific delivery. As an example, the smallest dose of 0.05 BAU will use one 4×4 mm square, while a larger dose of 0.5 BAU can include ten 4×4 mm squares. During manufacture, the size or dose could be controlled through surface area or thickness of the allergen delivery squares (or any other suitable shape).

An exemplary design is discussed below. To obtain 500 4×4 mm squares of 0.05 BAU an entire 100×80 mm collector will be coated with 25 BAU. Due to environmental disturbances it can be expected that a loss of about 20% can be expected, therefore, a total of 31.25 BAUs would be electrospun so that a total ejection volume will be 625 μL.

The 50 BAU/mL allergen and polymer solution can be prepared by magnetic stirring of 1.75 mL of polymer mix, e.g., 0.05 mM PVP in ethanol with a 0.75 mL of 167 BAU/mL allergen dilution.

Multiple different allergens or allergen epitopes can be incorporated into the allergen/polymer mix at the same time, or a single allergen or allergen epitope can be incorporated into the allergen/polymer mix. When the single allergen or allergen epitope is included, the allergen or allergen epitope matrix can be deposited on top of another matrix on the patch or in a different area of the patch. One patch could therefore contain matrices of different allergens or allergen epitopes.

The one or more allergens used with the disclosed intradermal delivery patch that can be entrapped in the fibrous polymer matrix can be any allergen or allergen epitope that is suitable for use in therapy. For instance, cat allergens including Fel d 1, Fel d 2 and Fel d 4 could be used, also, dog allergens including Can f 1, Can f 2, Can f 3, Can f 4, Can f 5 and Can f 6 could be used. As other non-limiting examples, any allergens of the following items could also be used: insects such as mites, ants, caddisfly, cockroaches, deer flies, fleas, flies, mosquitoes and moths; pollens such as grass pollens, weed pollens, tree pollens, shrub pollens flower pollens, cultivated plant pollens; microorganism allergens such as fungus, smuts; epithelia allergens from various animals such as cants, cattle, dog, gerbil, goats, guinea pigs, hamsters, hogs, horses, mice, rabbit, rats, canaries, chickens, ducks, geese and parakeet; various ingestant allergies, such as plant foods (e.g. apples and carrots), animal foods (e.g. beef and pork), poultry products (e.g. egg whites, egg yolks), dairy products (e.g. milk), fish (e.g. catfish and lobster); nuts such as almonds, brazil nuts, cashews, coconuts, hazelnuts, peanuts, pecans and walnuts; other miscellaneous inhalants such as cotton linters, cottonseed, flaxseed, gum Arabic, gum karaya, gum tragacanth, kapok seeds, orris root, pyrethrum, silk and tobacco leaf; and mixtures thereof.

Further, any other available allergens could also be entrapped in the fibrous polymer matrix for any suitable therapies.

### Example 6

### Delivery of Antibodies

To determine the disclosed intradermal delivery patch's ability to encapsulate and deliver antibodies, Alexa Fluor 647 conjugated AffiniPure donkey anti-goat IgG heavy and light chains were obtained from Jackson Immunoresearch, 705-485, Lot#89919. The lyophilized powder was reconstituted in 2 mL of sterile HBSS, resulting in a concentration of 0.25 mg/mL. Although IgG antibody is discussed in this example, other antibodies including but not limited to: IgA, IgD, IgE and IgM could also be included with the disclosed intradermal delivery patch.

The electrospinning mixture was prepared at a volume ratio of 35% IgG solution to 65% 0.075 mM PVP in ethanol, 0.308 mL and 0.572 mL, respectively. The IgG/PVP mixture was ejected at a controlled rate of 20 μL/minute for a total volume of 600 μL. Given previous measurements, that about 80% of the total volume deposits on an 8×10 cm collector plate, it was estimated that about 42 μg of Alexa IgG was present per plate or about 84 ng per 4×4 mm wafer. Subsequently, an IgG/PVP patch was created that consisted of twenty 4×4 wafers, consisting of about 1.68 μg of IgG.

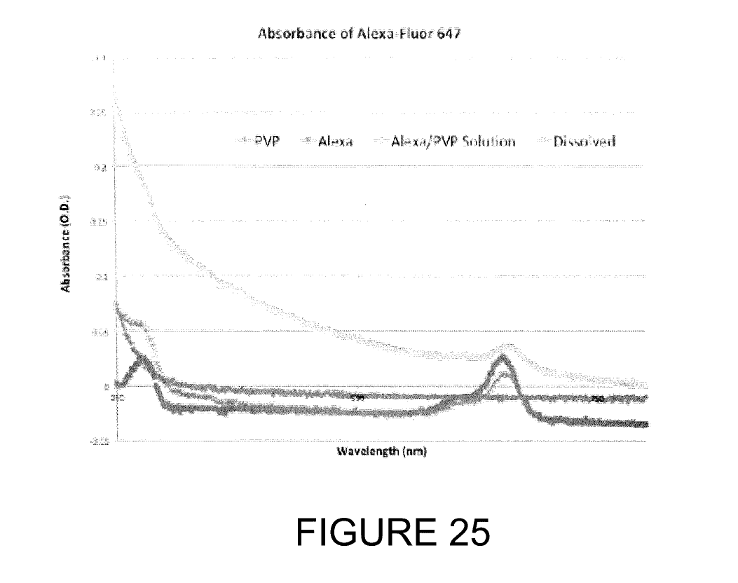

To determine if the antibody was fluorescently detectable throughout the electrospinning process, the relative absorbance across wavelengths 250-800 nm was measured. The absorbance value of stock AlexaIgG was compared to that of PVP solution, AlexaIgG/PVP solution and the dissolved AlexaIgG/PVP matrix. There appeared to be little or no change no change in absorbance of Alexa at the 647 nm wavelength, as shown in FIG. 25. Outside of the presence of hydrocarbon peaks at the lower wavelengths, there was little or no shift in the expected AlexaIgG peak.

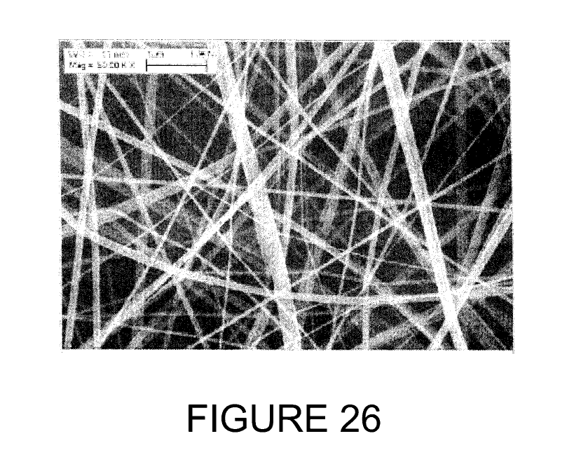

The structural integrity of the matrix was then examined. The AlexaIgG/PVP mixture, when electrospun at above outlined parameters, resulted in a nonwoven nanofibrous matrix, characterized by a relatively continuous porosity and an average fiber diameter of about 60 nm, as shown in FIG. 26, which are SEM images of the matrix.

To determine the patch's ability for AlexaIgG delivery, the patch was placed onto a rat's back for 24 hours. After 24 hours and euthanasia, 8 mm punch biopsies were collected in the area where the patch was located and were placed into a Petri dish with a drop of PBS so as to not dehydrate the samples. The samples were then evaluated with a table top fluorescent microscope probe, capable of penetrating the tissue. Any other suitable antibodies could also be delivered in a similar way.

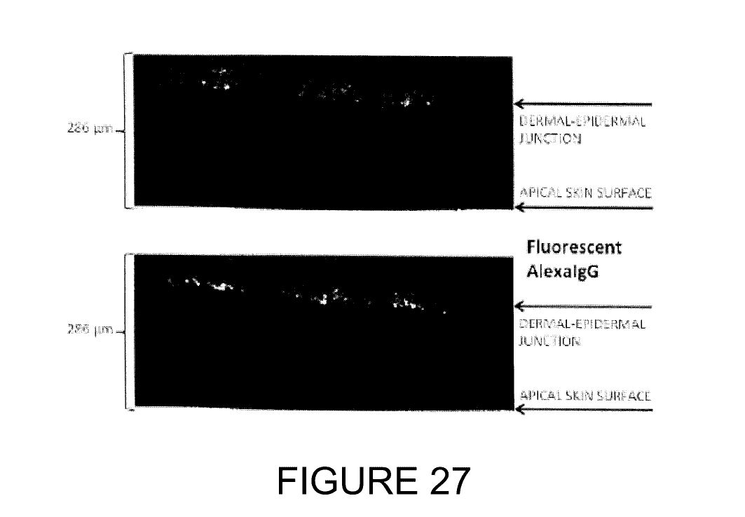

The inverted microscope images shown in FIG. 27 indicate the presence of the AlexaIgG approximately 200 μm from the skin's apical surface in the measured biopsies.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

What is claimed is:

1. An intradermal delivery patch comprising at least one antigen encapsulated by at least one fibrous polymer matrix.

2. The intradermal delivery patch of claim 1, wherein the at least one antigen is selected from the group consisting of influenza virus proteins, anthrax, pertussis toxin, and combinations thereof.

3. The intradermal delivery patch of claim 1, wherein the at least one antigen is combined with at least one adjuvant.

4. The intradermal immunization patch of claim 1, wherein the fibrous polymer matrix is hygroscopic.

5. The intradermal delivery patch of claim 1, wherein the fibrous polymer matrix is polyvinylpyrrolidone.

6. The intradermal delivery patch of claim 1, wherein the average molecular weight of the polyvinylpyrrolidone is about 100,000 g/mol to about 2,500,000 g/mol.

7. The intradermal delivery patch of claim 1, wherein the average molecular weight of the polyvinylpyrrolidone is 1,300,000 g/mol.

8. The intradermal delivery patch of claim 1, wherein the average diameter of the fibers is a diameter from about 10 nm to about 500 nm.

9. The intradermal delivery patch of claim 1, wherein the average diameter of the fibers is about 40 nm.

10. The intradermal delivery patch of claim 1, wherein the average diameter of the fibers is about 72 nm.

11. The intradermal delivery patch of claim 1, wherein the stability of the at least one antigen is improved by the encapsulation by the fibrous matrix.

12. A method for manufacturing the intradermal delivery patch of claim 1, comprising the steps of:
a. mixing a solution of at least one antigen and with a solution of a polymer; and
b. forming a fibrous non-woven membrane from the mixture.

13. The method of claim 12, wherein the fibrous membrane is formed by electrospinning.

14. The method of claim 12, wherein the fibrous membrane is formed on a silicon substrate.

15. A method for immunizing an animal using the intradermal delivery patch of claim 1, comprising the steps of:
a. attaching the at least one patch the onto the skin of the animal;
b. allowing the at least one patch to increase the hydration of the skin, thereby increasing the permeability of the skin to the at least one antigen; and
c. allowing the at least one patch to release the at least one antigen and enter the skin of the animal, thereby immunizing the animal.

16. The method of claim 15, wherein an occlusive dressing is applied over the at least one patch after the at least one patch is applied to the skin of the animal.

17. The method of claim 15, wherein the at least one patch releases 100%, 90%, 80%, 70%, 60%, 50%, 40%, 30%, 20%, or 10% of the at least one antigen encapsulated within the at least one patch when the at least one patch is attached to the skin of an animal.

18. The method of claim 15, wherein the at least one patch releases 100%, 90%, 80%, 70%, 60%, 50%, 40%, 30%, 20%, or 10% of the at least one antigen encapsulated within at least one patch within 24 hours after the at least one patch is attached to the skin of an animal.

19. The method of claim 15, wherein the at least one patch releases the at least one antigen to an absorbance of greater than 35 A.U. after 24 hours.

20. The method of claim 15, wherein the at least one patch attaches to the skin without mechanical disruption to the skin.

21. The method of claim 15, wherein the step of allowing the at least one patch to increase the hydration of the skin and the step of allowing the at least one patch to release the at least one antigen occur without addition of hydration to the patch.

22. The method of claim 15, wherein the animal is a human.

23. A method for delivering allergens to an animal using the intradermal delivery patch of claim 1, comprising the steps of:
a. attaching the at least one patch the onto the skin of the animal;
b. allowing the at least one patch to increase the hydration of the skin, thereby increasing the permeability of the skin to the at least one allergen; and
c. allowing the at least one patch to release the at least one allergen and enter the skin of the animal.

24. The method of claim 23, wherein an occlusive dressing is applied over the at least one patch after the at least one patch is applied to the skin of the animal.

25. The method of claim 23, wherein the at least one patch attaches to the skin without mechanical disruption to the skin

26. The method of claim 23, wherein the animal is a human.
